(19) United States
(12) Patent Application Publication
(10) Pub. No.: US 2022/0411876 A1
(43) Pub. Date: Dec. 29, 2022

(54) METHODS AND RELATED ASPECTS FOR ANALYZING MOLECULAR RESPONSE

(71) Applicant: GUARDANT HEALTH, INC., Redwood City, CA (US)

(72) Inventors: Katie Julia QUINN, Redwood City, CA (US); Allysia Jade MAK, Redwood City, CA (US); Elena HELMAN, Redwood City, CA (US); Tingting JIANG, Redwood City, CA (US); Justin I. ODEGAARD, Redwood City, CA (US); Darya CHUDOVA, San Jose, CA (US); Kyle Lik Ming CHANG, Seattle, WA (US); Han-Yu CHUANG, Redwood City, CA (US); Daniel GAILE, Redwood City, CA (US)

(21) Appl. No.: 17/687,536

(22) Filed: Mar. 4, 2022

Related U.S. Application Data
(60) Provisional application No. 63/173,193, filed on Apr. 9, 2021.
(60) Provisional application No. 63/157,592, filed on Mar. 5, 2021.

(51) Int. Cl. C12Q 1/6886; G16B 30/00; G16B 40/00

(52) U.S. Cl. CPC C12Q 1/6886; G16B 30/00; G16B 40/00; C12Q 2600/106; C12Q 2600/156; C12Q 1/6869

(57) ABSTRACT

Provided herein are methods of determining a molecular response score. The molecular response score may be used to monitor and guide administration of treatment to a subject.

### CROSS-REFERENCE TO RELATED APPLICATIONS

This application claims the benefit of priority of U.S. Provisional Patent Application No. 63/157,592, filed Mar. 5, 2021, and U.S. Provisional Patent Application No. 63/173,193, filed Apr. 9, 2021, each of which is incorporated by reference herein in its entirety for all purposes.

### BACKGROUND

Molecular response is a calculation of the change in circulating tumor DNA (ctDNA) levels observed in samples collected from subjects at different time points. In certain cases, the calculation is based on the fraction of somatic variants in the total cell-free DNA (cfDNA) in samples. In other cases, the calculation is based on the concentration of ctDNA in the samples (i.e., normalized per the cfDNA concentration in the samples). A common problem associated with these approaches is that these relatively simple calculations of molecular response frequently yield inaccurate or imprecise molecular response scores. Thus, there remains a need for methods for accurately determining molecular response scores for subjects having cancer.

### BRIEF SUMMARY

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads and the second plurality of sequence reads as somatic or germline, determining, for at least one variant of the plurality of variants classified as somatic, based on a first mutant allele fraction (MAF) and a second MAF, a weighted mean of the first MAFs and a weighted mean of the second MAFs, determining, for the subject, a ratio of the weighted mean of the first MAFs and the weighted mean of the second MAFs, determining, based on the ratio of the weighted mean of the first MAFs and the weighted mean of the second MAFs, a confidence interval, and outputting, as a molecular response score, the ratio of the weighted mean of the first MAFs and the weighted mean of the second MAFs and the confidence interval.

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads and the second plurality of sequence reads as somatic or germline, determining, for at least one variant of the plurality of variants classified as somatic, based on a first mutant allele fraction (MAF) and a second MAF, an MAF ratio, determining, for the subject, a weighted mean of the MAF ratios, determining, based on the weighted mean of the MAF ratios, a confidence interval associated with the weighted mean of the MAF ratios, and outputting, as a molecular response score, the weighted mean of the MAF ratios and the confidence interval.

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads as somatic or germline, classifying the plurality of variants in the second plurality of sequence reads as somatic or germline, reclassifying at least one variant of the plurality of variants to resolve a classification discrepancy between the first plurality of sequence reads and the second plurality of sequence reads, determining, for at least one variant of the plurality of variants classified or reclassified as somatic, based on at least a portion of the first plurality of sequence reads, a first mutant allele fraction, determining, for at least one variant of the plurality of variants classified or reclassified as somatic, based on at least a portion of the second plurality of sequence reads, a second mutant allele fraction, and determining, based on the first mutant allele fraction and the second mutant allele fraction, a molecular response score.

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads and the second plurality of sequence reads as somatic or germline, determining at least one variant of the plurality of variants as a Clonal Hematopoiesis of Indeterminate Potential (CHIP) variant, removing, from the plurality of variants, the at least one CHIP variant, determining, for at least one variant of the plurality of variants classified as somatic, based on at least a portion of the first plurality of sequence reads, a first mutant allele fraction, determining, for at least one variant of the plurality of variants classified as somatic, based on at least a portion of the second plurality of sequence reads, a second mutant allele fraction, and determining, based on the first mutant allele fraction and the second mutant allele fraction, a molecular response score.

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads as somatic or germline, classifying the plurality of variants in the second plurality of sequence reads as somatic or germline, reclassifying at least one variant of the plurality of variants to resolve a classification discrepancy between the first plurality of sequence reads and the second plurality of sequence reads, determining at least one variant of the plurality of variants as a Clonal Hematopoiesis of Indeterminate Potential (CHIP) variant, removing, from the plurality of variants, the at least one CHIP variant, determining, for at least one variant of the plurality of variants classified or reclassified as somatic, based on at least a portion of the first plurality of sequence reads, a first mutant allele fraction, determining, for at least one variant of the plurality of variants classified or reclassified as somatic, based on at least a portion of the second plurality of sequence reads, a second mutant allele fraction, determining, for at least one variant of the plurality of variants classified or reclassified as somatic, based on the first mutant allele fraction and the second mutant allele fraction, an MAF ratio, determining, for the subject, a weighted mean of the MAF ratios, determining, based on the weighted mean of the MAF ratios, a confidence interval associated with the weighted mean of the MAF ratios, and outputting, as a molecular response score, the weighted mean of the MAF ratios and the confidence interval.

In an aspect, this disclosure provides a method of determining a molecular response score at least partially using a computer. The method includes determining a first plurality of sequence reads and a second plurality of sequence reads associated with a subject, wherein the first plurality of sequence reads are determined before administering a therapy and the second plurality of sequence reads are determined after administering the therapy, classifying a plurality of variants in the first plurality of sequence reads as somatic or germline, classifying the plurality of variants in the second plurality of sequence reads as somatic or germline, reclassifying at least one variant of the plurality of variants to resolve a classification discrepancy between the first plurality of sequence reads and the second plurality of sequence reads, determining at least one variant of the plurality of variants as a Clonal Hematopoiesis of Indeterminate Potential (CHIP) variant, removing, from the plurality of variants, the at least one CHIP variant, determining, for at least one variant of the plurality of variants classified as somatic, based on at least a portion of the first plurality of sequence reads, a first mutant allele fraction (MAF), determining, for at least one variant of the plurality of variants classified as somatic, based on at least a portion of the second plurality of sequence reads, a second MAF, determining, for the at least one variant of the plurality of variants classified as somatic, based on the first MAF and the second MAF, a weighted mean of the first MAFs and a weighted mean of the second MAFs, determining, for the subject, a ratio of the weighted mean of the first MAFs and the weighted mean of the second MAFs, determining, based on the ratio of the weighted mean of the first MAFs and the weighted mean of the second MAFs, a confidence interval, and outputting, as a molecular response score, the ratio of the weighted mean of the first MAFs and the weighted mean of the second MAFs and the confidence interval.

In an aspect, this disclosure provides a method of determining a molecular response score at least partially using a computer. The method includes determining a first plurality of sequence reads and a second plurality of sequence reads associated with a subject, wherein the first plurality of sequence reads are determined at a first time point before administering a therapy and the second plurality of sequence reads are determined at a second time point after administering the therapy, classifying a plurality of variants in the first plurality of sequence reads and the second plurality of sequence reads as somatic or germline, determining, for at least one variant of the plurality of variants classified as somatic, based on a first mutant allele fraction (MAF) at the first time point and a second MAF at the second time point, a first central tendency measure of the first MAFs and a second central tendency measure of the second MAFs, determining a ratio of the first central tendency measure at the first time point to the second central tendency measure at the second time point, and outputting, as a molecular response score, the ratio of the first central tendency measure at the first time point to the second central tendency measure at the second time point.

In one aspect, this disclosure provides a method of determining a molecular response score for a subject having cancer at least partially using a computer. The method includes (a) determining, by the computer, mutant allele frequencies (MAFs) for a plurality of variants from sequence information generated from targeted nucleic acids associated with one or more cancer types in samples obtained from the subject at first and second time points to produce sets of first and second MAFs for each variant in the plurality of variants. The method also includes (b) calculating, by the computer, a ratio of the first and second MAFs for each variant in the plurality of variants to produce a set of MAF ratios and a corresponding standard deviation for each MAF ratio in the set of MAF ratios. In addition, the method also includes (c) calculating, by the computer, a weighted mean of the MAF ratios and a confidence interval, thereby determining the molecular response score for the subject having the cancer.

In another aspect, this disclosure provides a method of treating cancer in a subject. The method includes (a) determining mutant allele frequencies (MAFs) for a plurality of variants from sequence information generated from targeted nucleic acids associated with one or more cancer types in samples obtained from the subject at first and second time points to produce sets of first and second MAFs for each variant in the plurality of variants. The method also includes (b) calculating a ratio of the first and second MAFs for each variant in the plurality of variants to produce a set of MAF ratios and a corresponding standard deviation for each MAF ratio in the set of MAF ratios. The method also includes (c) calculating a weighted mean of the MAF ratios and a confidence interval to determine a molecular response score for the subject. In addition, the method also includes (d) administering one or more therapies to the subject based upon at least the molecular response score, thereby treating the cancer in the subject.

In another aspect, this disclosure provides a method of treating cancer in a subject. The method includes administering one or more therapies to the subject based upon at least a molecular response score for the subject. The molecular response score is produced by: (a) determining, by a computer, mutant allele frequencies (MAFs) for a plurality of variants from sequence information generated from targeted nucleic acids associated with one or more cancer types in samples obtained from the subject at first and second time points to produce sets of first and second MAFs for each variant in the plurality of variants; (b) calculating, by the computer, a ratio of the first and second MAFs for each variant in the plurality of variants to produce a set of MAF ratios and a corresponding standard deviation for each MAF ratio in the set of MAF ratios; and (c) calculating, by the computer, a weighted mean of the MAF ratios and a confidence interval to determine the molecular response score for the subject.

In another aspect, this disclosure provides a method of identifying clonal hematopoietic variants in a subject having cancer at least partially using a computer. The method includes (a) determining, by the computer, a tumor load change (R) for tumor fraction change P(R) for each of a plurality of variants from sequence information generated from targeted nucleic acids associated with one or more cancer types in samples obtained from the subject at first and second time points to produce a set of tumor load changes. The method also includes (b) identifying, by the computer, one or more resistance signatures corresponding to one or more clonal hematopoietic variants from the set of tumor load changes, thereby identifying the identifying the clonal hematopoietic variants in the subject having cancer.

In another aspect, this disclosure provides a method of identifying clonal hematopoietic variants in a subject having cancer at least partially using a computer. The method includes (a) calculating, by the computer, a probability density function for tumor fraction change P(R) for each of a plurality of variants from sequence information generated from targeted nucleic acids associated with one or more cancer types in samples obtained from the subject at first and second time points. The method also includes (b) grouping, by the computer, one or more of the variants by P(R) into one or more clones, and (c) generating, by the computer, an updated P(R) for each of the clones. In addition, the method also includes (d) identifying, by the computer, one or more clones having a fractional change between the first and second time points at or above a predetermined threshold value, thereby identifying the identifying the clonal hematopoietic variants in the subject having cancer. In some of these embodiments, the method includes determining a likelihood that a given pair of variants exhibit an identical fractional change, merging most likely pairs of variants into one clone, and updating the P(R) for the one clone.

In another aspect, this disclosure provides a method of identifying germline variants in a subject having cancer at least partially using a computer. The method includes (a) determining, by the computer, a mutant allele frequency (MAF) for a given variant from sequence information generated from targeted nucleic acids associated with one or more cancer types in a sample obtained from the subject. The method also includes (b) identifying, by the computer, that the given variant is a germline variant when the MAF of the given variant increases the max MAF of the sample, which sample comprises a max fraction of diploid genes (max frac_diploid) and/or when the MAF of the given variant is at least about two times greater, three times greater, four times greater, five times greater, six times greater, seven times greater, eight times greater, nine times greater, or more than one or more other MAFs determined from the sample obtained from the subject, thereby identifying the germline variants in the subject having cancer.

In some embodiments, the methods disclosed herein include comparing the molecular response score for the subject having the cancer to a predetermined cutoff point to identify that the subject is a likely responder to one or more therapies for the cancer when the molecular response score is below the predetermined cutoff point or that the subject is a likely non-responder to the one or more therapies for the cancer when the molecular response score is at or above the predetermined cutoff point. In some embodiments, the one or more therapies comprise one or more immunotherapies. In some embodiments, the methods disclosed herein include administering one or more therapies for the cancer to the subject in view of the molecular response score. In some embodiments, the methods disclosed herein include discontinuing administering one or more therapies for the cancer to the subject in view of the molecular response score. In some embodiments, the methods disclosed herein include recommending one or more therapies. In some embodiments, the methods disclosed herein include recommending discontinuing one or more therapies. In some embodiments, the methods disclosed herein include using the molecular response score as a prognostic biomarker and/or a predictive biomarker for the subject.

In some embodiments, the methods disclosed herein include using a molecule count to calculate the standard deviation for each MAF ratio in the set of MAF ratios. In some embodiments, the methods disclosed herein include propagating a variance through each MAF ratio in the set of MAF ratios. In some embodiments, the methods disclosed herein include excluding one or more germline and/or clonal hematopoietic variants when determining the mutant allele frequencies (MAFs) for the plurality of variants. In some embodiments, the plurality of variants comprises somatic nucleic acid variants. In some embodiments, the methods disclosed herein include excluding one or more somatic variants having MAFs that are less than about 0.1%, 0.2%, 0.3%, 0.4%, 0.5%, 0.6%, 0.7%, 0.8%, or 0.9% at both the first and second time points. In some embodiments, the first time point comprises a pre-treatment time point and wherein the second time point comprises an on- or post-treatment time point.

In some embodiments, the methods disclosed herein include generating the sequence information from nucleic acid molecules obtained from one or more tissues or cells in the sample. In some embodiments, the methods disclosed herein include generating the sequence information from cell-free nucleic acids (cfNAs) in the samples obtained from the subject. In some embodiments, the cfNAs comprise circulating tumor DNA (ctDNA).

In some embodiments, the ratio comprises the second MAF to the first MAF for each variant in the plurality of variants. In some embodiments, the methods disclosed herein include calculating the weighted mean of the MAF ratios using the formula:

sum[weight*ratio]/sum[weights],

where weight is 1/range² for a given variant in the plurality of variants, where range is a difference between values of the first and second MAFs for a given variant in the plurality of variants, and ratio is a given MAF ratio in the set of MAF ratios. In some embodiments, the methods disclosed herein include calculating the confidence interval using the formula:

weighted mean of the MAF ratios+/−sqrt[ratio variance],

where ratio variance is 1/sum[weights].

In some embodiments, the variants comprise one or more single-nucleotide variants (SNV), insertion/deletion mutations (indels), gene amplifications, and/or gene fusions. In some embodiments, the methods disclosed herein include using one or more additional genomic data sources to determine the molecular response score for the subject having the cancer. In some embodiments, the additional genomic data sources comprise one or more of: a coverage, an off-target coverage, an epigenetic signature, and/or a microsatellite instability score. In some embodiments, the epigenetic signature comprises a cfNA fragment length, position, and/or endpoint density distribution. In some embodiments, the epigenetic signature comprises an epigenetic state or status exhibited by one or more epigenetic loci in a given targeted genomic region. In some embodiments, the epigenetic state or status comprises a presence or absence of methylation, hydroxymethylation, acetylation, ubiquitylation, phosphorylation, sumoylation, ribosylation, citrullination, and/or a histone post-translational modification or other histone variation.

This application discloses methods, computer readable media, and systems that are useful in determining molecular response scores for subjects having cancer. Related methods of identifying clonal hematopoietic and/or germline variants are also disclosed. Additional advantages of the disclosed method, systems, and/or compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an example method.

FIG. 2 shows an example method.

FIG. 3 shows an example method.

FIG. 4 shows an example method.

FIG. 5 shows an example method.

FIG. 6A shows an example method.

FIG. 6B shows an example method.

FIG. 7 shows an example method.

FIG. 8 shows an example method.

FIG. 9 shows an example method.

FIG. 10 shows an example method.

FIG. 11 shows an example method.

FIG. 12A shows an example method.

FIG. 12B shows an example method.

FIG. 13 shows an example method.

FIG. 14 shows an example method.

FIG. 15 shows an example method.

FIG. 16 shows an example method.

FIG. 17 shows an example method.

FIG. 18 shows an example method.

FIG. 19 shows an example method.

FIG. 20 shows an example method.

FIG. 21 shows an example system.

FIG. 22 shows the number of somatic variants detected per sample in a panel space.

FIG. 23 shows an example of somatic classification discrepancies that could skew MR results.

FIGS. 24A-24F show an example of variant precision determined by Mutant Molecule Count (MMC=VAF*Molecular Coverage). (A) Variants have a range of molecular coverage, depending on sample input and panel design. Probability of variant detection (B) and VAF precision (C) depends on both VAF and molecular coverage (colors, mapping to (A)). MMC (D) is a better metric for variant precision, because it determines the probability of variant detection (E). VAF precision (F).

FIGS. 25A-25C shows that tumor signal can be outweighed by a minority of variants when using Mean of ratios, m(rVAF), or ratio of max, R(maxVAF). (A) MR score is categorized as Increasing, Decreasing or within precision limit (“Near 0% Change”). (B) Shows patient molecular response score by method. (C) Graph of R(mVAF) only baseline evaluable variants (Y-axis) versus R(mVAF) all evaluable variants. Dark circles are evaluable; lighter circles (seen in a line across the x-axis) are not evaluable.

FIGS. 26A-26C show an example that certainty in molecular response score increases with increasing number of variants (A), molecular coverage (B), and maximum VAF (C).

FIGS. 27A and 27B show a histogram of molecular response scores for clinical samples (A) and technical replicates (null distribution) (B), with hypothetical examples of variant trajectories.

FIG. 28 shows an example determination of a molecular response score.

### DETAILED DESCRIPTION

The disclosed method and compositions may be understood more readily by reference to the following detailed description of particular embodiments and the Examples included therein and to the Figures and their previous and following description.

It is to be understood that the disclosed method and compositions are not limited to specific synthetic methods, specific analytical techniques, or to particular reagents unless otherwise specified, and, as such, may vary.

### I. Definitions

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth. It will also be appreciated that there is an implied “about” prior to the temperatures, concentrations, times, number of bases or base pairs, coverage, etc. discussed in the present disclosure, such that slight and insubstantial equivalents are within the scope of the present disclosure. In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Adapter: As used herein, “adapter” refers to short nucleic acids (e.g., less than about 500, less than about 100 or less than about 50 nucleotides in length) that are typically at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequencing reads of a given nucleic acid molecule. The same or different adapters can be linked to the respective ends of a nucleic acid molecule. In certain embodiments, the same adapter is linked to the respective ends of the nucleic acid molecule except that the nucleic acid tag differs. In some embodiments, the adapter is a Y-shaped adapter in which one end is blunt ended or tailed as described herein, for joining to a nucleic acid molecule, which is also blunt ended or tailed with one or more complementary nucleotides. In still other exemplary embodiments, an adapter is a bell-shaped adapter that includes a blunt or tailed end for joining to a nucleic acid molecule to be analyzed. Other exemplary adapters include T-tailed and C-tailed adapters.

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Allele: As used herein, “allele” or “allelic variant” refers to a specific genetic variant at defined genomic location or locus. An allelic variant is usually presented at a frequency of 50% (0.5) or 100%, depending on whether the allele is heterozygous or homozygous. For example, germline variants are inherited and usually have a frequency of 0.5 or 1. Somatic variants; however, are acquired variants and usually have a frequency of <0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

Amplify: As used herein, “amplify” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, “barcode” in the context of nucleic acids refers to a nucleic acid molecule comprising a sequence that can serve as a molecular identifier. For example, individual “barcode” sequences are typically added to each DNA fragment during next-generation sequencing (NGS) library preparation so that each read can be identified and sorted before the final data analysis.

Cancer Type: As used herein, “cancer,” “cancer type” or “tumor type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Cell-Free Nucleic Acid: As used herein, “cell-free nucleic acid” refers to nucleic acids not contained within or otherwise bound to a cell or, in some embodiments, nucleic acids remaining in a sample following the removal of intact cells. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Cell-free nucleic acids can be found in an efferosome or an exosome. Some cell-free nucleic acids are released into bodily fluid from cancer cells, e.g., circulating tumor DNA (ctDNA). Others are released from healthy cells. CtDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Classifier: As used herein, “classifier” generally refers to algorithm computer code that receives, as input, test data and produces, as output, a classification of the input data as belonging to one or another class (e.g., tumor DNA or non-tumor DNA).

Clonal: As used herein, “clonal” in the context of nucleic acids refers to a population of nucleic acids that comprises nucleotide sequences that are substantially or completely identical to each other at least at a given locus of interest (e.g., a target variant).

Clonal Hematopoiesis of Indeterminate Potential: As used herein, “clonal hematopoiesis of indeterminate potential,” “clonal hematopoietic variant,” or “CHIP” refers to hematopoiesis in individuals that involves the expansion of hematopoietic stem cells that comprise one or more somatic mutations (e.g., hematologic cancer-associated mutations and/or non-cancer-associated mutations), but which otherwise lack diagnostic criteria for a hematologic malignancy, such as definitive morphologic evidence of dysplasia. CHIP is a common age-related phenomenon in which hematopoietic stem cells contribute to the formation of a genetically distinct subpopulation of blood cells.

Confidence Interval: As used herein, “confidence interval” or “level of confidence” means a range of values so defined that there is a specified probability that the value of a given parameter lies within that range of values.

Copy Number Variant: As used herein, “copy number variant,” “CNV,” or “copy number variation” refers to a phenomenon in which sections of the genome are repeated and the number of repeats in the genome varies between individuals in the population under consideration.

Coverage: As used herein, “coverage” refers to the number of nucleic acid molecules that represent a particular base position.

Deoxyribonucleic Acid or Ribonucleic Acid: As used herein, “deoxyribonucleic acid” or “DNA” refers a natural or modified nucleotide which has a hydrogen group at the 2′-position of the sugar moiety. DNA typically includes a chain of nucleotides comprising four types of nucleotide bases: adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA typically includes a chain of nucleotides comprising four types of nucleotide bases: A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “sequence information,” “nucleic acid sequence,” “nucleotide sequence”, “genomic sequence,” “genetic sequence,” or “fragment sequence,” or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Detect: As used herein, “detect,” “detecting,” or “detection” refers to an act of determining the existence or presence of one or more target nucleic acids (e.g., nucleic acids having targeted mutations or other markers) in a sample.

Enriched Sample: As used herein, “enriched sample” refers to a sample that has been enriched for specific regions of interest. The sample can be enriched by amplifying regions of interest or by using single-stranded DNA/RNA probes or double stranded DNA probes that can hybridize to nucleic acid molecules of interest (e.g., SureSelect® probes, Agilent Technologies). In some embodiments, an enriched sample refers to a subset or portion of the processed sample that is enriched, where the subset or portion of the processed sample being enriched contains nucleic acid molecules from a sample of cell-free polynucleotides or polynucleotides.

Epigenetic Information: As used herein, “epigenetic information” in the context of a DNA polymer means one or more epigenetic patterns or signatures exhibited in that polymer.

Epigenetic Locus: As used herein, “epigenetic locus” or “epigenetic site” means a fixed position on a chromosome that exhibits different states or statuses that do not involve changes or alterations in nucleotide sequence. For the avoidance of doubt, a given epigenetic locus can coincide with a given nucleotide position or genomic region that also exhibits genetic or sequence variation (e.g., mutations). For example, a given epigenetic locus may or may not be acetylated, methylated (e.g., modified with 5-methylcytosine (5mC), modified with 5-hydroxymethylcytosine (5hmC), and/or the like), ubiquitylated, phosphorylated, sumoylated, ribosylated, citrullinated, have a histone post-translational modification or other histone variation, and/or the like.

Epigenetic Signature: As used herein, “epigenetic signature” means an epigenetic state or status exhibited by one or more epigenetic loci in a given DNA molecule. For example, DNA molecules or cfDNA fragments that comprise a given genomic region or locus (e.g., a CTCF binding region, etc.) may also exhibit epigenetic patterns in which some of those DNA molecules include a certain number of epigenetic loci that are methylated, whereas in other instances corresponding epigenetic loci in other DNA molecules or cfDNA fragments that comprise the same genomic region are unmethylated.

Germline Mutation: As used herein, “germline mutation” means a mutation in nucleic acids in a germ cell that is present prior to conception.

Immunotherapy: As used herein, “immunotherapy” refers to treatment with one or more agents that act to stimulate the immune system so as to kill or at least to inhibit growth of cancer cells, and preferably to reduce further growth of the cancer, reduce the size of the cancer and/or eliminate the cancer. Some such agents bind to a target present on cancer cells; some bind to a target present on immune cells and not on cancer cells; some bind to a target present on both cancer cells and immune cells. Such agents include, but are not limited to, checkpoint inhibitors and/or antibodies. Checkpoint inhibitors are inhibitors of pathways of the immune system that maintain self-tolerance and modulate the duration and amplitude of physiological immune responses in peripheral tissues to minimize collateral tissue damage (see, e.g., Pardoll, Nature Reviews Cancer 12, 252-264 (2012)). Exemplary agents include antibodies against any of PD-1, PD-2, PD-L1, PD-L2, CTLA-4, OX40, B7.1, B7He, LAG3, CD137, KIR, CCR5, CD27, CD40, or CD47. Other exemplary agents include proinflammatory cytokines, such as IL-1β, IL-6, and TNF-α. Other exemplary agents are T-cells activated against a tumor, such as T-cells activated by expressing a chimeric antigen targeting a tumor antigen recognized by the T-cell.

Indel: As used herein, “indel” refers to mutation that involves the insertion or deletion of nucleotide positions in the genome of a subject.

Maximum Mutant Allele Frequency: As used herein, “maximum mutant allele frequency,” “maximum variant allele frequency,” “maximum MAF,” “MAX MAF,” “maximum VAF,” “max-MAF” or “MAX VAF” refers to the maximum or largest MAF of all somatic variants present or observed in a given sample.

Mutant Allele Frequency: As used herein, “mutant allele frequency,” “variant allele frequency,” “mutant allele fraction,” “variant allele fraction,” “MAF,” or “VAF” refers to the frequency at which mutant alleles occur in a given population of nucleic acids, such as a sample obtained from a subject. MAF is generally expressed as a fraction or a percentage.

Molecular Response: As used herein, “molecular response” refers to a change in one or more circulating tumor DNA (ctDNA) variant allele frequencies, levels, or amounts observed in between samples taken from a given subject at different time points.

Molecular Responder: As used herein, “molecular responder” or “responder” refers to a subject having a molecular response score that indicates a decrease in one or more circulating tumor DNA (ctDNA) variant allele frequencies, levels, or amounts observed in between samples taken from the subject at different time points.

Molecular Non-Responder: As used herein, “molecular non-responder” or “non-responder” refers to subject having a molecular response score that indicates an increase, or no change, in one or more circulating tumor DNA (ctDNA) variant allele frequencies, levels, or amounts observed in between samples taken from the subject at different time points. A threshold specifying a level of decrease (or increase) may be utilized to determine whether the subject is a molecular responder or a molecular non-responder. For example, a molecular responder may be a subject associated with a decrease of more than a certain percentage change in VAF, and a non-responder may be a subject associated with an increase, or no change, or a decrease by less than a certain percentage change in VAF.

Mutation: As used herein, “mutation,” “nucleic acid variant,” “variant,” or “genetic aberration” refers to a variation from a known reference sequence and includes mutations such as, for example, single nucleotide variants (SNVs), copy number variants or variations (CNVs)/aberrations, insertions or deletions (indels), truncation, gene fusions, transversions, translocations, frame shifts, duplications, repeat expansions, and epigenetic variants. A mutation can be a germline or somatic mutation. In some embodiments, a reference sequence for purposes of comparison is a wildtype genomic sequence of the species of the subject providing a test sample, typically the human genome. In certain cases, a mutation or variant is a “tumor-related genetic variant” that causes or at least contributes to oncogenesis.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization.

Nucleic Acid Tag: As used herein, “nucleic acid tag” refers to a short nucleic acid (e.g., less than about 500, about 100, about 50 or about 10 nucleotides in length), used to label nucleic acid molecules to distinguish nucleic acids from different samples (e.g., representing a sample index), or different nucleic acid molecules in the same sample (e.g., representing a molecular tag), of different types, or which have undergone different processing. Nucleic acid tags can be single stranded, double stranded or at least partially double stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or both ends of the other nucleic acids (e.g., sample nucleic acids to be amplified and/or sequenced). Nucleic acid tags can be decoded to reveal information such as the sample of origin, form or processing of a given nucleic acid. Nucleic acid tags can also be used to enable pooling and/or parallel processing of multiple samples comprising nucleic acids bearing different nucleic acid tags and/or sample indexes in which the nucleic acids are subsequently being deconvoluted by reading the nucleic acid tags. Nucleic acid tags can also be referred to as molecular identifiers or tags, sample identifiers, index tags, and/or barcodes. Additionally or alternatively, nucleic acid tags can be used to distinguish different molecules in the same sample. This includes, for example, uniquely tagging each different nucleic acid molecule in a given sample, or non-uniquely tagging such molecules. In the case of non-unique tagging applications, a limited number of tags may be used to tag each nucleic acid molecule such that different molecules can be distinguished based on, for example, start/stop positions where they map to a selected reference genome in combination with at least one nucleic acid tag. Typically, a sufficient number of different nucleic acid tags are used such that there is a low probability (e.g., less than about a 10%, less than about a 5%, less than about a 1%, or less than about a 0.1% chance) that any two molecules will have the same start/stop positions and also have the same nucleic acid tag. Some nucleic acid tags include multiple molecular identifiers to label samples, forms of nucleic acid molecules within a sample, and nucleic acid molecules within a form having the same start and stop positions. Such nucleic acid tags can be referenced using the exemplary form “Ali” in which the uppercase letter indicates a sample type, the Arabic numeral indicates a form of molecule within a sample, and the lowercase Roman numeral indicates a molecule within a form.

Polynucleotide: As used herein, “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases, as is standard in the art.

Reference Sample: As used herein, “reference sample” or “reference cfNA sample” refers to a sample of known composition and/or having or known to have or lack specific properties (e.g., known nucleic acid variant(s), known cellular origin, known tumor fraction, known coverage, and/or the like) that is analyzed along with or compared to test samples in order to evaluate the accuracy of an analytical procedure, classify the test samples, and/or the like. A reference sample dataset typically includes from at least about 25 to at least about 30,000 or more reference samples. In some embodiments, the reference sample dataset includes about 50, 75, 100, 150, 200, 300, 400, 500, 600, 700, 800, 900, 1,000, 2,500, 5,000, 7,500, 10,000, 15,000, 20,000, 25,000, 50,000, 100,000, 1,000,000, or more reference samples.

Reference Sequence: As used herein, “reference sequence” or “reference genome” refers to a known sequence used for purposes of comparison with experimentally determined sequences. For example, a known sequence can be an entire genome, a chromosome, or any segment thereof. A reference sequence typically includes at least about 20, at least about 50, at least about 100, at least about 200, at least about 250, at least about 300, at least about 350, at least about 400, at least about 450, at least about 500, at least about 1000, or more nucleotides. A reference sequence can align with a single contiguous sequence of a genome or chromosome or can include non-contiguous segments that align with different regions of a genome or chromosome. Exemplary reference sequences, include, for example, human genomes, such as, hG19 and hG38.

Sample: As used herein, “sample” means any biological sample capable of being analyzed by the methods and/or systems disclosed herein. In certain aspects of the present disclosure, samples are bodily fluid samples, for example, whole blood or fractions thereof, lymphatic fluid, urine, and/or cerebrospinal fluid, among other bodily fluid types from which cell-free (circulating, not contained within or otherwise bound to a cell) nucleic acids are sourced. In certain implementations, bodily fluid samples are plasma samples, which are the fluid portions of whole blood exclusive of cells, such as red and white blood cells. In some implementations, bodily fluid samples are serum samples, that is, plasma lacking fibrinogen. In some aspects of the present disclosure, samples are “non-bodily fluid samples” or “non-plasma samples,” that is, biological samples other than “bodily fluid samples” such as, as cellular and/or tissue samples, from which nucleic acids other than cell-free nucleic acids are sourced.

Sensitivity: As used herein, “sensitivity” in the context of a given assay or method refers to the ability of the assay or method to detect and distinguish between targeted (e.g., cfDNA fragments originating from tumor cells) and non-targeted (e.g., cfDNA fragments originating from non-tumor cells) analytes.

Sequencing: As used herein, “sequencing” refers to any of a number of technologies used to determine the sequence (e.g., the identity and order of monomer units) of a biomolecule, e.g., a nucleic acid such as DNA or RNA. Exemplary sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

Single Nucleotide Variant: As used herein, “single nucleotide variant” or “SNV” means a mutation or variation in a single nucleotide that occurs at a specific position in the genome.

Somatic Mutation: As used herein, “somatic mutation” means a mutation in the genome that occurs after conception. Somatic mutations can occur in any cell of the body except germ cells and accordingly, are not passed on to progeny.

Specificity: As used herein, “specificity” in the context of a diagnostic analysis or assay refers to the extent to which the analysis or assay detects an intended target analyte to the exclusion of other components of a given sample.

Sub-Clonal: As used herein, “sub-clonal” in the context of nucleic acids refers to a sub-population of nucleic acids (i.e., a subset of the population of nucleic acids) that comprises nucleotide sequences that are substantially or completely identical to each other at least at a given locus of interest (e.g., a target variant). For example, sub-clonal can refer to a subset of cancer cells.

Subject: As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

Threshold Value: As used herein, “threshold value” refers to a separately determined value used to characterize or classify experimentally determined values.

Tumor Fraction: As used herein, “tumor fraction” refers to the estimate of the fraction of nucleic acid molecules derived from tumor in a given sample. For example, the tumor fraction of a sample can be a measure derived from the maximum somatic mutant allele frequency (max MAF) of the sample or coverage of the sample, or length, epigenetic state, or other properties of the cfNA fragments in the sample or any other selected feature of the sample. In some embodiments, the tumor fraction of a sample is equal to the max MAF of the sample.

Value: As used herein, “value” generally refers to an entry in a dataset can be anything that characterizes the feature to which the value refers. This includes, without limitation, numbers, words or phrases, symbols (e.g., + or −) or degrees.

Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Where the invention provides a process involving multiple sequential steps, the invention can also provide a process where these different steps can be performed at very different times by different people in different places (e.g. in different countries).

### II. Molecular Response Scoring

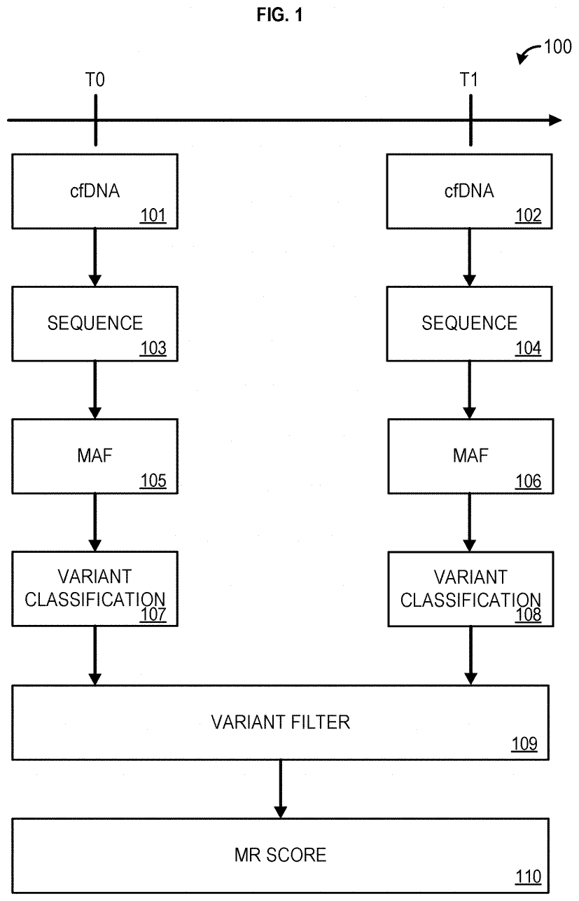

In an embodiment, shown in FIG. 1, a method 100 for determining a Molecular response (MR) score is disclosed. The methods of this disclosure may have a wide variety of uses in the manipulation, preparation, identification, quantification, and/or analysis of cell-free nucleic acids. Molecular response is an assessment of the change in circulating tumor DNA (ctDNA) load on-treatment (usually 3-10 weeks) in comparison to pre-treatment baseline. Molecular response is associated with patient response to therapy and long term outcomes across solid tumors and therapy types. Molecular response can also be used to predict clinical response earlier than radiographic and/or RECIST response. Multiple methods have been used to calculate molecular response and there is no consensus regarding which method is best.

Methods and systems are described for assessing response to treatment using a molecular response (MR) score. In an embodiment, baseline (pre-treatment) gene expression data may be obtained for a plurality of patients prior to treatment and on-treatment gene expression data may be obtained for the plurality of patients during treatment. In an embodiment, the baseline gene expression data (e.g., variant data) and/or the on-treatment gene expression data may be analyzed to determine a molecular response (MR) score. The MR score may indicate that a patient is a responder or a non-responder to the treatment. In an embodiment, a mutant allele fraction (MAF) may be determined as part of the MR score. In an embodiment, the variance of each MAF may be incorporated into the determination of the molecular response score. This ensures molecular response scores include accurate variance, which provides a significant improvement in making a correct conclusion from the molecular response score. The improvement is even more pronounced when the molecular response score is a ratio, as a ratio is sensitive to variance in the denominator. The variance can be incorporated into the molecular response score either through deriving mathematically the molecular response variance or through simulation or sampling from the variance distribution of each variant to determine the molecular response variance.

a. cfDNA Isolation and Extraction

As shown in FIG. 1, at a first time T₀, baseline cfDNA may be obtained from one or more baseline samples obtained from one or more subjects prior to treatment at step 101 and at a second time T₁, on-treatment cfDNA may be obtained from one or more on-treatment samples obtained from one or more subjects after treatment at step 102. Treatment may occur/being at any time subsequent to time T₀. For example, treatment may occur minutes, hours, days, etc. after time T₀. By way of further example, treatment may occur 30 minutes after time T₀, 1 hour to 2 hours after time T₀, 1 day to 2 days after time T₀, 1 week to 2 weeks after time T₀, 1 month to 2 months after time T₀, 6 months to 1 year after time T₀, 1 year to 2 years after time T₀, and the like. Time T₁ can be any amount of time after time T₀, for example, any time between and including 1-24 hours, 1-180 days, 1-12 weeks, 6-12 months, and the like.

As described herein, a polynucleotide can comprise any type of nucleic acid, such as DNA and/or RNA. For example, if a polynucleotide is DNA, it can be genomic DNA, complementary DNA (cDNA), or any other deoxyribonucleic acid. A polynucleotide can also be a cell-free nucleic acid such as cell-free DNA (cfDNA). For example, the polynucleotide can be circulating cfDNA. Circulating cfDNA may comprise DNA shed from bodily cells via apoptosis or necrosis. cfDNA shed via apoptosis or necrosis may originate from normal (e.g. healthy) bodily cells. Where there is abnormal tissue growth, such as for cancer, tumor DNA may be shed. The circulating cfDNA can comprise circulating tumor DNA (ctDNA).

i. Samples

Isolation and extraction of cell free polynucleotides may be performed through collection of samples using a variety of techniques. A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid sample for analysis is plasma or serum containing cell-free nucleic acids, e.g., cell-free DNA (cfDNA).

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled blood is typically between about 5 ml to about 20 ml.

The sample can comprise various amounts of nucleic acid. Typically, the amount of nucleic acid in a given sample is equated with multiple genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some embodiments, a sample comprises nucleic acids from different sources, e.g., from cells and from cell-free sources (e.g., blood samples, etc.). Typically, a sample includes nucleic acids carrying mutations. For example, a sample optionally comprises DNA carrying germline mutations and/or somatic mutations. Typically, a sample comprises DNA carrying cancer-associated mutations (e.g., cancer-associated somatic mutations). In some embodiments of the present disclosure, cell free nucleic acids in a subject may derive from a tumor. For example cell-free DNA isolated from a subject can comprise ctDNA.

Exemplary amounts of cell-free nucleic acids in a sample before amplification typically range from about 1 femtogram (fg) to about 1 microgram (μg), e.g., about 1 picogram (pg) to about 200 nanogram (ng), about 1 ng to about 100 ng, about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning step in which cell-free nucleic acids, as found in solution, are separated from intact cells and other non-soluble components of the bodily fluid. In some of these embodiments, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain embodiments, additional clean up steps are used, such as silica-based columns to remove contaminants or salts. Non-specific bulk carrier nucleic acids, for example, are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps. Additional details regarding cfDNA partitioning and related analysis of epigenetic modifications that are optionally adapted for use in performing the methods disclosed herein are described in, for example, WO 2018/119452, filed Dec. 22, 2017, which is incorporated by reference.

ii. Nucleic Acid Tags

In certain embodiments, tags providing molecular identifiers or barcodes are incorporated into or otherwise joined to adapters by chemical synthesis, ligation, or overlap extension PCR, among other methods. In some embodiments, the assignment of unique or non-unique identifiers, or molecular barcodes in reactions follows methods and utilizes systems described in, for example, US patent applications 20010053519, 20030152490, 20110160078, and U.S. Pat. Nos. 6,582,908, 7,537,898, and 9,598,731, which are each incorporated by reference.

Tags are linked (e.g., ligated) to sample nucleic acids randomly or non-randomly. In some embodiments, tags are introduced at an expected ratio of identifiers (e.g., a combination of unique and/or non-unique barcodes) to microwells. For example, the identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some embodiments, the identifiers are loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In certain embodiments, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample. The identifiers are generally unique or non-unique.

One exemplary format uses from about 2 to about 1,000,000 different tags, or from about 5 to about 150 different tags, or from about 20 to about 50 different tags, ligated to both ends of a target nucleic acid molecule. For 20-50×20-50 tags, a total of 400-2500 tags are created. Such numbers of tags are typically sufficient for different molecules having the same start and stop points to have a high probability (e.g., at least 94%, 99.5%, 99.99%, 99.999%) of receiving different combinations of tags.

In some embodiments, identifiers are predetermined, random, or semi-random sequence oligonucleotides. In other embodiments, a plurality of barcodes may be used such that barcodes are not necessarily unique to one another in the plurality. In these embodiments, barcodes are generally attached (e.g., by ligation or PCR amplification) to individual molecules such that the combination of the barcode and the sequence it may be attached to creates a unique sequence that may be individually tracked. As described herein, detection of non-uniquely tagged barcodes in combination with sequence data of beginning (start) and end (stop) portions of sequence reads typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

iii. Nucleic Acid Amplification

Sample nucleic acids flanked by adapters are typically amplified by PCR and other amplification methods using nucleic acid primers binding to primer binding sites in adapters flanking a DNA molecule to be amplified. In some embodiments, amplification methods involve cycles of extension, denaturation and annealing resulting from thermocycling, or can be isothermal as, for example, in transcription mediated amplification. Other exemplary amplification methods that are optionally utilized, include the ligase chain reaction, strand displacement amplification, nucleic acid sequence-based amplification, and self-sustained sequence-based replication, among other approaches.

One or more rounds of amplification cycles are generally applied to introduce sample indexes/tags to a nucleic acid molecule using conventional nucleic acid amplification methods. The amplifications are typically conducted in one or more reaction mixtures. In some embodiments, molecular tags and sample indexes/tags are introduced prior to and/or after sequence capturing steps are performed. In some embodiments, only the molecular tags are introduced prior to probe capturing and the sample indexes/tags are introduced after sequence capturing steps are performed. In certain embodiments, both the molecular tags and the sample indexes/tags are introduced prior to performing probe-based capturing steps. In some embodiments, the sample indexes/tags are introduced after sequence capturing steps (i.e., enrichment of nucleic acids) are performed. Typically, sequence capturing protocols involve introducing a single-stranded nucleic acid molecule complementary to a targeted nucleic acid sequence, e.g., a coding sequence of a genomic region and mutation of such region associated with a cancer type. Typically, the amplification reactions generate a plurality of non-uniquely or uniquely tagged nucleic acid amplicons with molecular tags and sample indexes/tags at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

iv. Nucleic Acid Enrichment

In some embodiments, sequences are enriched prior to sequencing the nucleic acids. Enrichment is optionally performed for specific target regions or nonspecifically (“target sequences”). By way of example, enrichment may be performed nonspecifically based on a size selection method that is not sequence specific but rather is sequence fragment size specific. In some embodiments, targeted regions of interest may be enriched with nucleic acid capture probes (“baits”) selected for one or more bait set panels using a differential tiling and capture scheme. A differential tiling and capture scheme generally uses bait sets of different relative concentrations to differentially tile (e.g., at different “resolutions”) across genomic sections associated with the baits, subject to a set of constraints (e.g., sequencer constraints such as sequencing load, utility of each bait, etc.), and capture the targeted nucleic acids at a desired level for downstream sequencing. These targeted genomic sections of interest optionally include natural or synthetic nucleotide sequences of the nucleic acid construct. In some embodiments, biotin-labeled beads with probes to one or more sections of interest can be used to capture target sequences, and optionally followed by amplification of those sections, to enrich for the regions of interest.

Sequence capture typically involves the use of oligonucleotide probes that hybridize to the target nucleic acid sequence. In certain embodiments, a probe set strategy involves tiling the probes across a section of interest. Such probes can be, for example, from about 60 to about 120 nucleotides in length. The set can have a depth of about 2×, 3×, 4×, 5×, 6×, 8×, 9×, 10×, 15×, 20×, 50× or more. The effectiveness of sequence capture generally depends, in part, on the length of the sequence in the target molecule that is complementary (or nearly complementary) to the sequence of the probe.

b. Nucleic Acid Sequencing

As shown in FIG. 1, after extraction and isolation of cfDNA from samples at steps 101 and 102, the cfDNA may be sequenced at steps 103 and 104. Sample nucleic acids, optionally flanked by adapters, with or without prior amplification are generally subject to sequencing. Sequencing methods or commercially available formats that are optionally utilized include, for example, Sanger sequencing, high-throughput sequencing, bisulfate sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, primer walking, sequencing using PacBio, SOLiD, Ion Torrent, or nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one more nucleic acid fragment types or sections known to contain markers of cancer or of other diseases. The sequencing reactions can also be performed on any nucleic acid fragment present in the sample. The sequence reactions may provide for sequence coverage of the genome of at least about 5%, 10%, 15%, 20%, 25%, 30%, 40%, 50%, 60%, 70%, 80%, 90%, 95%, 99%, 99.9% or 100% of the genome. In other cases, sequence coverage of the genome may be less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some embodiments, cell-free polynucleotides are sequenced with at least about 1000, 2000, 3000, 4000, 5000, 6000, 7000, 8000, 9000, 10000, 50000, or 100,000 sequencing reactions. In other embodiments, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some embodiments, data analysis is performed on at least about 1000, 2000, 3000, 4000, 5000, 6000, 7000, 8000, 9000, 10000, 50000, or 100,000 sequencing reactions. In other embodiments, data analysis may be performed on less than about 1000, 2000, 3000, 4000, 5000, 6000, 7000, 8000, 9000, 10000, 50000, or 100,000 sequencing reactions. An exemplary read depth is from about 1000 to about 50000 reads per locus (base position).

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U). Exemplary enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including barcodes, and the sequencing determines nucleic acid sequences as well as in-line barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (e.g., sticky end ligation).

The nucleic acid sample is typically contacted with a sufficient number of adapters such that there is a low probability (e.g., <1 or 0.1%) that any two copies of the same nucleic acid receive the same combination of adapter barcodes from the adapters linked at both ends. The use of adapters in this manner permits identification of families of nucleic acid sequences with the same start and stop points on a reference nucleic acid and linked to the same combination of barcodes. Such a family represents sequences of amplification products of a template/parent nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence may be eliminated from subsequent analysis.

Nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, the length of a given cfDNA fragment based upon where its endpoints (i.e., it 5′ and 3′ terminal nucleotides) map to the reference sequence, the offset of a midpoint of a given cfDNA fragment from a midpoint of a genomic region in the cfDNA fragment, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Additional details regarding nucleic acid sequencing, including the formats and applications described herein are also provided in, for example, Levy et al., Annual Review of Genomics and Human Genetics, 17: 95-115 (2016), Liu et al., J. of Biomedicine and Biotechnology, Volume 2012, Article ID 251364:1-11 (2012), Voelkerding et al., Clinical Chem., 55: 641-658 (2009), MacLean et al., Nature Rev. Microbiol., 7: 287-296 (2009), Astier et al., J Am Chem Soc., 128(5):1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

i. Sequencing Panel

To improve the likelihood of detecting genomic regions of interest and optionally, tumor indicating mutations, the sections of DNA sequenced may comprise a panel of genes or genomic sections that comprise known genomic regions. Selection of a limited section for sequencing (e.g., a limited panel) can reduce the total sequencing needed (e.g., a total amount of nucleotides sequenced). A sequencing panel can target a plurality of different genes or regions, for example, to detect a single cancer, a set of cancers, or all cancers. Alternatively, DNA may be sequenced by whole genome sequencing (WGS) or other unbiased sequencing method without the use of a sequencing panel. Examples of suitable panel and targets for use in panels can be found in the epigenetic targets described in U.S. provisional patent application 62/799,637, filed Jan. 31, 2019, which is incorporated by reference in its entirety.

In some aspects, a panel that targets a plurality of different genes or genomic regions (e.g., transcriptional factor binding regions, distal regulatory elements (DREs), repetitive elements, intron-exon junctions, transcriptional start sites (TSSs), and/or the like) is selected such that a determined proportion of subjects having a cancer exhibits a genetic variant or tumor marker in one or more different genes in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. The panel can comprise a plurality of subpanels, including subpanels for identifying tissue of origin (e.g., use of published literature to define 50-100 baits representing genes with most diverse transcription profile across tissues (not necessarily promoters)), whole genome scaffold (e.g., for identifying ultra-conservative genomic content and tiling sparsely across chromosomes with handful of probes for copy number base lining purposes), transcription start site (TSS)/CpG islands (e.g., for capturing differential methylated regions (e.g., Differentially Methylated Regions (DMRs)) in for example in promoters of tumor suppressor genes (e.g., SEPT9/VIM in colorectal cancer)). In some embodiments, markers for a tissue of origin are tissue-specific epigenetic markers.

Some examples of listings of genomic locations of interest may be found in Table 1 and Table 2. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or 97 of the genes of Table 1. In an embodiment, genomic locations used in the methods of the present disclosure comprise all genes of Table 1. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, or 70 of the SNVs of Table 1. In an embodiment, genomic locations used in the methods of the present disclosure comprise all SNVs of Table 1. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 1. In an embodiment, genomic locations used in the methods of the present disclosure comprise all CNVs of Table 1. In some embodiments, genomic locations used in the methods of the present disclosure comprise at least 1, at least 2, at least 3, at least 4, at least 5, or 6 of the fusions of Table 1. In an embodiment, genomic locations used in the methods of the present disclosure comprise all fusions of Table 1. In some embodiments, genomic locations used in the methods of the present disclosure comprise at least a portion of at least 1, at least 2, or 3 of the indels of Table 1. In an embodiment, genomic locations used in the methods of the present disclosure comprise all indels of Table 1. In an embodiment, genomic locations used in the methods of the present disclosure comprise all genes, SNVs, CNVs, fusions, and indels of Table 1. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, or 115 of the genes of Table 2. In an embodiment, genomic locations used in the methods of the present disclosure comprise all genes of Table 2. In an embodiment, genomic locations used in the methods of the present disclosure comprise all genes of Table 1 and Table 2. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, at least 70, or 73 of the SNVs of Table 2. In an embodiment, genomic locations used in the methods of the present disclosure comprise all SNVs of Table 2. In an embodiment, genomic locations used in the methods of the present disclosure comprise all SNVs of Table 1 and Table 2. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 2. In an embodiment, genomic locations used in the methods of the present disclosure comprise all CNVs of Table 2. In an embodiment, genomic locations used in the methods of the present disclosure comprise all CNVs of Table 1 and Table 2. In some embodiments, genomic locations used in the methods of the present disclosure comprise at least 1, at least 2, at least 3, at least 4, at least 5, or 6 of the fusions of Table 2. In an embodiment, genomic locations used in the methods of the present disclosure comprise all fusions of Table 2. In an embodiment, genomic locations used in the methods of the present disclosure comprise all fusions of Table 1 and Table 2. In some embodiments, genomic locations used in the methods of the present disclosure comprise at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 2. In an embodiment, genomic locations used in the methods of the present disclosure comprise all indels of Table 2. In an embodiment, genomic locations used in the methods of the present disclosure comprise all indels of Table 1 and Table 2. In an embodiment, genomic locations used in the methods of the present disclosure comprise all genes, SNVs, CNVs, fusions, and indels of Table 2. In an embodiment, genomic locations used in the methods of the present disclosure comprise all genes, SNVs, CNVs, fusions, and indels of Table 1 and Table 2. Each of these genomic locations of interest may be identified as a backbone region or hot-spot region for a given bait set panel.

TABLE 1

| | | | | | | Amplifications | | | |
|---|---|---|---|---|---|---|---|---|---|
| Point Mutations (SNVs) | | | | | | (CNVs) | | Fusions | Indels |
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | CDKN2B | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | |
| RIT1 | ROS1 | SMAD4 | SMO | SRC | STK11 | | | | |
| TERT | TP53 | TSC1 | VHL | | | | | | |

TABLE 2

| | | | | | | Amplifications | | | |
|---|---|---|---|---|---|---|---|---|---|
| Point Mutations (SNVs) | | | | | | (CNVs) | | Fusions | Indels |
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | DDR2 | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | ATM |
| RIT1 | ROS1 | SMAD4 | SMO | MAPK1 | STK11 | | | | |
| TERT | TP53 | TSC1 | VHL | MAPK3 | MTOR | | | | |
| NTRK3 | | | | | | | | | APC |

ARID1A
BRCA1
BRCA2
CDH1
CDKN2A
GATA3
KIT
MLH1
MTOR
NF1
PDGFRA
PTEN
RB1
SMAD4
STK11
TP53
TSC1
VHL

In some embodiments, the one or more regions in the panel comprise one or more loci from one or a plurality of genes for detecting residual cancer after surgery. This detection can be earlier than is possible for existing methods of cancer detection. In some embodiments, the one or more genomic locations in the panel comprise one or more loci from one or a plurality of genes for detecting cancer in a high-risk patient population. For example, smokers have much higher rates of lung cancer than the general population. Moreover, smokers can develop other lung conditions that make cancer detection more difficult, such as the development of irregular nodules in the lungs. In some embodiments, the methods described herein detect the response of patients to cancer therapy (particularly in high risk patients) earlier than is possible for existing methods of cancer detection.

A genomic location may be selected for inclusion in a sequencing panel based on a number of subjects with a cancer that have a tumor marker in that gene or region. A genomic location may be selected for inclusion in a sequencing panel based on prevalence of subjects with a cancer and a tumor marker present in that gene. Presence of a tumor marker in a region may be indicative of a subject having cancer.

In some instances, the panel may be selected using information from one or more databases. The information regarding a cancer may be derived from cancer tumor biopsies or cfDNA assays. A database may comprise information describing a population of sequenced tumor samples. A database may comprise information about mRNA expression in tumor samples. A database may comprise information about regulatory elements or genomic regions in tumor samples. The information relating to the sequenced tumor samples may include the frequency of various genetic variants and describe the genes or regions in which the genetic variants occur. The genetic variants may be tumor markers. A non-limiting example of such a database is COSMIC. COSMIC is a catalogue of somatic mutations found in various cancers. For a particular cancer, COSMIC ranks genes based on frequency of mutation. A gene may be selected for inclusion in a panel by having a high frequency of mutation within a given gene. For instance, COSMIC indicates that 33% of a population of sequenced breast cancer samples have a mutation in TP53 and 22% of a population of sampled breast cancers have a mutation in KRAS. Other ranked genes, including APC, have mutations found only in about 4% of a population of sequenced breast cancer samples. TP53 and KRAS may be included in a sequencing panel based on having relatively high frequency among sampled breast cancers (compared to APC, for example, which occurs at a frequency of about 4%). COSMIC is provided as a non-limiting example, however, any database or set of information may be used that associates a cancer with tumor marker located in a gene or genetic region. In another example, as provided by COSMIC, of 1156 biliary tract cancer samples, 380 samples (33%) carried mutations in TP53. Several other genes, such as APC, have mutations in 4-8% of all samples. Thus, TP53 may be selected for inclusion in the panel based on a relatively high frequency in a population of biliary tract cancer samples.

A gene or genomic section may be selected for a panel where the frequency of a tumor marker is significantly greater in sampled tumor tissue or circulating tumor DNA than found in a given background population. A combination of genomic locations may be selected for inclusion of a panel such that at least a majority of subjects having a cancer may have a tumor marker or genomic region present in at least one of the genomic location or genes in the panel. The combination of genomic location may be selected based on data indicating that, for a particular cancer or set of cancers, a majority of subjects have one or more tumor markers in one or more of the selected regions. For example, to detect cancer 1, a panel comprising regions A, B, C, and/or D may be selected based on data indicating that 90% of subjects with cancer 1 have a tumor marker in regions A, B, C, and/or D of the panel. Alternately, tumor markers may be shown to occur independently in two or more regions in subjects having a cancer such that, combined, a tumor marker in the two or more regions is present in a majority of a population of subjects having a cancer. For example, to detect cancer 2, a panel comprising regions X, Y, and Z may be selected based on data indicating that 90% of subjects have a tumor marker in one or more regions, and in 30% of such subjects a tumor marker is detected only in region X, while tumor markers are detected only in regions Y and/or Z for the remainder of the subjects for whom a tumor marker was detected. Tumor markers present in one or more genomic locations previously shown to be associated with one or more cancers may be indicative of or predictive of a subject having cancer if a tumor marker is detected in one or more of those regions 50% or more of the time. Computational approaches such as models employing conditional probabilities of detecting cancer given a cancer frequency for a set of tumor markers within one or more regions may be used to predict which regions, alone or in combination, may be predictive of cancer. Other approaches for panel selection involve the use of databases describing information from studies employing comprehensive genomic profiling of tumors with large panels and/or whole genome sequencing (WGS, RNA-seq, Chip-seq, bisulfate sequencing, ATAC-seq, and others). Information gleaned from literature may also describe pathways commonly affected and mutated in certain cancers. Panel selection may be further informed by the use of ontologies describing genetic information.

Genes included in the panel for sequencing can include the fully transcribed region, the promoter region, enhancer regions, regulatory elements, and/or downstream sequence. To further increase the likelihood of detecting tumor indicating mutations only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

In some aspects, a panel of exons from each of a plurality of different genes is selected such that a determined proportion of subjects having a cancer exhibit a genetic variant in at least one exon in the panel of exons.

At least one full exon from each different gene in a panel of genes may be sequenced. The sequenced panel may comprise exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be at least 10 kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, or 150 kb in size. The sequencing panel may be less than 100 kb, 90 kb, 80 kb, 70 kb, 60 kb, or 50 kb in size.

The panel selected for sequencing can comprise at least 1, 5, 10, 15, 20, 25, 30, 40, 50, 60, 80, or 100 genomic locations (e.g., that each include genomic regions of interest). In some cases, the genomic locations in the panel are selected that the size of the locations are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the genomic locations in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel selected herein can allow for deep sequencing that is sufficient to detect low-frequency genetic variants (e.g., in cell-free nucleic acid molecules obtained from a sample). An amount of genetic variants in a sample may be referred to in terms of the mutant allele frequency for a given genetic variant. The mutant allele frequency may refer to the frequency at which mutant alleles (e.g., not the most common allele) occurs in a given population of nucleic acids, such as a sample. Genetic variants at a low mutant allele frequency may have a relatively low frequency of presence in a sample. In some cases, the panel allows for detection of genetic variants at a mutant allele frequency of at least 0.0001%, 0.001%, 0.005%, 0.01%, 0.05%, 0.1%, or 0.5%. The panel can allow for detection of genetic variants at a mutant allele frequency of 0.001% or greater. The panel can allow for detection of genetic variants at a mutant allele frequency of 0.01% or greater. The panel can allow for detection of genetic variant present in a sample at a frequency of as low as 0.0001%, 0.001%, 0.005%, 0.01%, 0.025%, 0.05%, 0.075%, 0.1%, 0.25%, 0.5%, 0.75%, or 1.0%. The panel can allow for detection of tumor markers present in a sample at a frequency of at least 0.0001%, 0.001%, 0.005%, 0.01%, 0.025%, 0.05%, 0.075%, 0.1%, 0.25%, 0.5%, 0.75%, or 1.0%. The panel can allow for detection of tumor markers at a frequency in a sample as low as 1.0%. The panel can allow for detection of tumor markers at a frequency in a sample as low as 0.75%. The panel can allow for detection of tumor markers at a frequency in a sample as low as 0.5%. The panel can allow for detection of tumor markers at a frequency in a sample as low as 0.25%. The panel can allow for detection of tumor markers at a frequency in a sample as low as 0.1%. The panel can allow for detection of tumor markers at a frequency in a sample as low as 0.075%. The panel can allow for detection of tumor markers at a frequency in a sample as low as 0.05%. The panel can allow for detection of tumor markers at a frequency in a sample as low as 0.025%. The panel can allow for detection of tumor markers at a frequency in a sample as low as 0.01%. The panel can allow for detection of tumor markers at a frequency in a sample as low as 0.005%. The panel can allow for detection of tumor markers at a frequency in a sample as low as 0.001%. The panel can allow for detection of tumor markers at a frequency in a sample as low as 0.0001%. The panel can allow for detection of tumor markers in sequenced cfDNA at a frequency in a sample as low as 1.0% to 0.0001%. The panel can allow for detection of tumor markers in sequenced cfDNA at a frequency in a sample as low as 0.01% to 0.0001%.

A genetic variant can be exhibited in a percentage of a population of subjects who have a disease (e.g., cancer). In some cases, at least 1%, 2%, 3%, 5%, 10%, 20%, 30%, 40%, 50%, 60%, 70%, 80%, 90%, 95%, or 99% of a population having the cancer exhibit one or more genetic variants in at least one of the regions in the panel. For example, at least 80% of a population having the cancer may exhibit one or more genetic variants in at least one of the genomic positions in the panel.

The panel can comprise one or more locations comprising genomic regions of interest from each of one or more genes. In some cases, the panel can comprise one or more locations comprising genomic regions of interest from each of at least 1, 2, 3, 4, 5, 6, 7, 8, 9, 10, 15, 20, 25, 30, 40, 50, or 80 genes. In some cases, the panel can comprise one or more locations comprising genomic regions of interest from each of at most 1, 2, 3, 4, 5, 6, 7, 8, 9, 10, 15, 20, 25, 30, 40, 50, or 80 genes. In some cases, the panel can comprise one or more locations comprising genomic regions of interest from each of from about 1 to about 80, from 1 to about 50, from about 3 to about 40, from 5 to about 30, from 10 to about 20 different genes.

The locations comprising genomic regions in the panel can be selected so that one or more epigenetically modified regions are detected. The one or more epigenetically modified regions can be acetylated, methylated, ubiquitylated, phosphorylated, sumoylated, ribosylated, and/or citrullinated. For example, the regions in the panel can be selected so that one or more methylated regions are detected.

The regions in the panel can be selected so that they comprise sequences differentially transcribed across one or more tissues. In some cases, the locations comprising genomic regions can comprise sequences transcribed in certain tissues at a higher level compared to other tissues. For example, the locations comprising genomic regions can comprise sequences transcribed in certain tissues but not in other tissues.

The genomic locations in the panel can comprise coding and/or non-coding sequences. For example, the genomic locations in the panel can comprise one or more sequences in exons, introns, promoters, 3′ untranslated regions, 5′ untranslated regions, regulatory elements, transcription start sites, and/or splice sites. In some cases, the regions in the panel can comprise other non-coding sequences, including pseudogenes, repeat sequences, transposons, viral elements, and telomeres. In some cases, the genomic locations in the panel can comprise sequences in non-coding RNA, e.g., ribosomal RNA, transfer RNA, Piwi-interacting RNA, and microRNA.

The genomic locations in the panel can be selected to detect (diagnose) a cancer with a desired level of sensitivity (e.g., through the detection of one or more genetic variants). For example, the regions in the panel can be selected to detect the cancer (e.g., through the detection of one or more genetic variants) with a sensitivity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the cancer with a sensitivity of 100%.

The genomic locations in the panel can be selected to detect (diagnose) a cancer with a desired level of specificity (e.g., through the detection of one or more genetic variants). For example, the genomic locations in the panel can be selected to detect cancer (e.g., through the detection of one or more genetic variants) with a specificity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the one or more genetic variant with a specificity of 100%.

The genomic locations in the panel can be selected to detect (diagnose) a cancer with a desired positive predictive value. Positive predictive value can be increased by increasing sensitivity (e.g., chance of an actual positive being detected) and/or specificity (e.g., chance of not mistaking an actual negative for a positive). As a non-limiting example, genomic locations in the panel can be selected to detect the one or more genetic variant with a positive predictive value of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a positive predictive value of 100%.

The genomic locations in the panel can be selected to detect (diagnose) a cancer with a desired accuracy. As used herein, the term “accuracy” may refer to the ability of a test to discriminate between a disease condition (e.g., cancer) and healthy condition. Accuracy may be can be quantified using measures such as sensitivity and specificity, predictive values, likelihood ratios, the area under the ROC curve, Youden's index and/or diagnostic odds ratio.

Accuracy may be presented as a percentage, which refers to a ratio between the number of tests giving a correct result and the total number of tests performed. The regions in the panel can be selected to detect cancer with an accuracy of at least 50%, 55%, 60%, 65%, 70%, 75%, 80%, 85%, 90%, 95%, 96%, 97%, 98%, 99%, 99.5%, or 99.9%. The genomic locations in the panel can be selected to detect cancer with an accuracy of 100%.

A panel may be selected to be highly sensitive and detect low frequency genetic variants. For instance, a panel may be selected such that a genetic variant or tumor marker present in a sample at a frequency as low as 0.01%, 0.05%, or 0.001% may be detected at a sensitivity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a sensitivity of 70% or greater. A panel may be selected to detect a tumor marker at a frequency in a sample as low as 0.1% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.01% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.001% with a sensitivity of at least 50%, 55%, 60%, 65%, 70%, 75%, 80%, 85%, 90%, 95%, 96%, 97%, 98%, 99%, 99.5%, or 99.9%.

A panel may be selected to be highly specific and detect low frequency genetic variants. For instance, a panel may be selected such that a genetic variant or tumor marker present in a sample at a frequency as low as 0.01%, 0.05%, or 0.001% may be detected at a specificity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a specificity of 70% or greater. A panel may be selected to detect a tumor marker at a frequency in a sample as low as 0.1% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with a specificity of at least 70%, 75%, 80%, 85%, 90%, 95%, 96%, 97%, 98%, 99%, 99.5%, or 99.9%.

A panel may be selected to be highly accurate and detect low frequency genetic variants. A panel may be selected such that a genetic variant or tumor marker present in a sample at a frequency as low as 0.01%, 0.05%, or 0.001% may be detected at an accuracy of at least 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with an accuracy of 70% or greater. A panel may be selected to detect a tumor marker at a frequency in a sample as low as 0.1% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with an accuracy of at least 70%, 75%, 80%, 85%, 90%, 95%, 96%, 97%, 98%, 99%, 99.5%, or 99.9%.

A panel may be selected to be highly predictive and detect low frequency genetic variants. A panel may be selected such that a genetic variant or tumor marker present in a sample at a frequency as low as 0.01%, 0.05%, or 0.001% may have a positive predictive value of at least 70%, 75%, 80%, 85%, 90%, 95%, 96%, 97%, 98%, 99%, 99.5%, or 99.9%.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances this may allow for more molecules within a biological to be analyzed thereby enabling lower frequency alleles to be detected.

In an embodiment, after sequencing, sequence reads may be assigned a quality score. A quality score may be a representation of sequence reads that indicates whether those sequence reads may be useful in subsequent analysis based on a threshold. In some cases, some sequence reads are not of sufficient quality or length to perform a subsequent mapping step. Sequence reads with a quality score at least 90%, 95%, 99%, 99.9%, 99.99% or 99.999% may be filtered out of a data set of sequence reads. In other cases, sequence reads assigned a quality scored at least 90%, 95%, 99%, 99.9%, 99.99% or 99.999% may be filtered out of the data set. Sequence reads that meet a specified quality score threshold may be mapped to a reference genome. After mapping alignment, sequence reads may be assigned a mapping score. A mapping score may be a representation of sequence reads mapped back to the reference sequence indicating whether each position is or is not uniquely mappable. Sequence reads with a mapping score at least 90%, 95%, 99%, 99.9%, 99.99% or 99.999% may be filtered out of the data set. In other cases, sequencing reads assigned a mapping scored less than 90%, 95%, 99%, 99.9%, 99.99% or 99.999% may be filtered out of the data set.

c. MAF Determination

As shown in FIG. 1, after cfDNA sequencing of samples at steps 103 and/or 104, one or more mutant allele fractions (MAFs) may be determined at steps 105 and/or 106. Some or all MAF determination may occur prior to variant classification 107/108, after variant classification 107/108, during variant classification 107/108, before variant filtering 109, after variant filtering 109, during variant filtering 109, or a combination thereof. Prior to step 103, cfDNA can be end repaired, ligated with adapters comprising molecular barcodes, amplified, and enriched. Amplification can incorporate sample index. In an embodiment, MAF values may be determined for all variants or all somatic variants. In an embodiment, MAF values may be determined for less than all variants or less than all somatic variants. Variant allele fraction (VAF) is used herein interchangeably with MAF. The mutant allele fraction (MAF) represents the number of mutant molecules divided by the total number of molecules (e.g., molecular coverage) at a specific genomic position:

$$MAF = \frac{Number of mutant molecules}{Total number of molecules}$$

A maximum MAF may be determined as the maximum or largest MAF of all somatic variants present or observed in a given sample. In some embodiments, maximum MAF can be considered as tumor fraction of a given sample.

A maximum fraction of diploid genes (“max frac_diploid”) (least allele imbalance) may be determined. A fraction of diploid genes (“frac_diploid) is a measure of the level of allele imbalance across the sample as determined by copy number. Samples with high levels of allele imbalance are prone to germline/somatic misclassification. Therefore, a low level of allele imbalance (or high frac_diploid) is an indication of the reliability of the somatic classification call.

In an embodiment, a total coverage profile may be used to capture fold change and thus tumor fraction, rather than individual genes.

d. Variant Classification

Sequencing at steps 103 and 104 generates a plurality of sequence reads. The plurality of sequence reads may be analyzed to determine one or more variants and to classify the one or more variants at steps 107 and/or 108. In an embodiment, some or all variant classification may be determined prior to MAF determination 105/106, after MAF determination 105/106, during MAF determination 105/106, or combinations thereof. Variants may include, for example, single nucleotide variants (SNV's), indels, fusions, and copy number variation. Any known technique for variant calling may be used. In an embodiment, the plurality of sequence reads from a sample may be assembled and/or mapped and aligned to genomic positions relative to a reference genome. In some embodiments, the plurality of sequence reads (assembled or otherwise) may then be compared to the reference genome to determine how the plurality of sequence reads of the subject vary from that of the reference genome. Such a process may determine the presence of one or more variants in the plurality of sequence reads. In some embodiments, the molecular barcodes and/or start and stop genomic positions of a nucleic acid molecule obtained from the plurality of sequence reads can be used to identify the mutant molecules where the sequence reads belonging to the molecule differ from the reference genome. Such a process may determine the presence of one or more variants in the plurality of sequence reads.

In an embodiment, common heterozygous SNPs may be used to model local germline allele count behavior and call variants somatic if they deviate significantly from observed germline mutant allele fraction. A betabinomial model may be used as it models both the mean and variance of mutant allele counts at common SNPs. For example, the betabinomial model described in PCT/US2018/052087, hereby incorporated by reference in its entirety, can be used. This is an improvement over simpler methods like fixed MAF cutoffs or Poisson models as they may not represent the variance in molecule counts appropriately.

e. Variant Filtering

In an embodiment, as shown in FIG. 1, one or more filtering processes may be applied at step 109 to the sequence reads to exclude sequence reads from further analysis. In an embodiment, some or all filtering may be applied prior to MAF determination 105/106, after MAF determination 105/106, during MAF determination 105/106, before variant classification 107/108, after variant classification 107/108, during variant classification 107/108, or a combination thereof.

In some embodiments, one or more somatic variants having MAFs that are less than about 0.1%, 0.2%, 0.3%, 0.4%, 0.5%, 0.6%, 0.7%, 0.8%, or 0.9% at the first and/or second time points may be excluded from further analysis. In some embodiments, one or more somatic variants having less than 5, 10, 15, 20, 25 or 30 mutant molecule counts at the first and/or second time points may be excluded from further analysis. In some embodiments, one or more somatic variants having a coverage less than 50, 100, 200, 300, 400, 500, 600, 700, 800, 900 or 1000 at the first and/or second time points may be excluded from further analysis.

In an embodiment, copy number variants may be used to exclude sequence reads from further analysis. Copy number amplifications may be determined as is known in the art. At step 109, the method 100 may filter out copy number amplifications in genes with either insufficient probe coverage or insufficient copy number (e.g. below the 95% limit of detection).

By way of example, CNVs may be determined by analyzing sequence reads to generate a chromosomal region of coverage. The chromosomal regions may be divided into variable length windows or bins. Read coverage may be determined for each window/bin region. In an embodiment, a quantitative measure related to sequencing read coverage is a measure indicative of the number of reads derived from a DNA molecule corresponding to a genetic locus (e.g., a particular position, base, region, gene or chromosome from a reference genome). In order to associate reads to a genetic locus, the reads can be mapped or aligned to the reference. Software to perform mapping or aligning (e.g., Bowtie, BWA, mrsFAST, BLAST, BLAT) can associate a sequencing read with a genetic locus. After the sequence read coverage has been determined, a stochastic modeling algorithm may be applied to convert the normalized nucleic acid sequence read coverage for each window/bin region to the discrete copy number states. In some cases, this algorithm may comprise one or more of the following: Hidden Markov Model, dynamic programming, support vector machine, Bayesian network, trellis decoding, Viterbi decoding, expectation maximization, Kalman filtering methodologies and neural networks. The discrete copy number states of each window region can be utilized to identify copy number variation in the chromosomal regions. In some cases, all adjacent window/bin regions with the same copy number can be merged into a segment to report the presence or absence of copy number variation state. In some cases, various windows/bins can be filtered before they are merged with other segments. Copy number variation may be used to report a percentage score indicating how much disease material (or nucleic acids having a copy number variation) exists in a cell free polynucleotide sample.

In an embodiment, the existence of CNVs in one or more genes may be used to exclude variants from further analysis. By way of example, variants having a threshold number of LDT-reportable genes with copy number >=a gene-specific 95% limit of detection (LoD) in either T₀ or T₁ sample. The threshold may be from about 10 to about 30. The threshold may be, for example, 15, 16, 17, 18, 19, 20, 21, 22, 23, 24, 25, etc. In an embodiment, the threshold may be 19.

Copy number variation may indicate fold change for a given variant. A Gaussian model may be used to determine a ratio of fold changes between time T₀ and time T₁ which may be used as an estimate of molecular response score.

In an embodiment, in the event the subject has no somatic variants, or has no variants that satisfy criteria of the variant filtering process, the subject may be classified as not-evaluable. In an embodiment, a subject classified as non-evaluable may be further classified as a molecular responder. In an embodiment, a subject having low ctDNA at both time T₀ and time T₁ may be classified as non-evaluable and further classified as a molecular responder. In an embodiment, a subject having a low MAF at both time T₀ and time T₁ may be classified as non-evaluable and further classified as a molecular responder. In an embodiment, a subject having a low tumor fraction at both time T₀ and time T₁ may be classified as non-evaluable and further classified as a molecular responder. Low MAF or low tumor fraction may refer to an MAF or a tumor fraction below a limit of detection (e.g. below the 95% limit of detection), or below a limit of quantification. What constitutes low may depend on panel design, but for example, an MAF f 0.1, 0.2, or 0.3% may be considered low.

i. Germline Filter

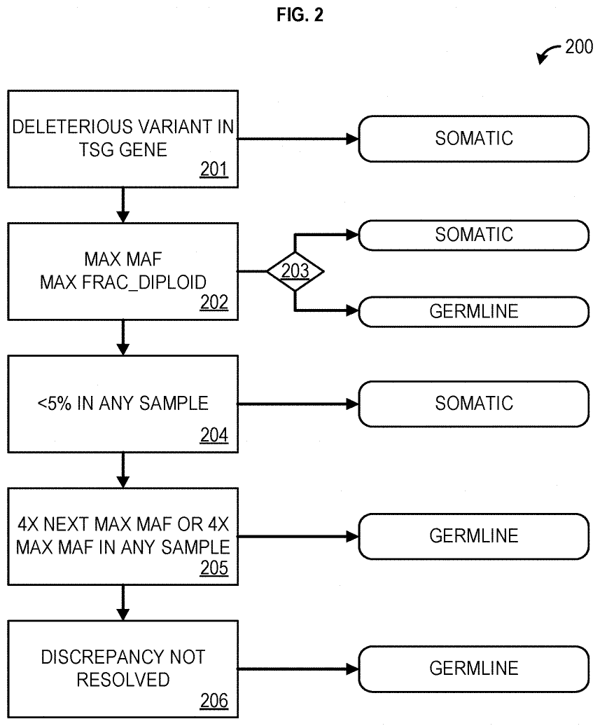

In an embodiment, shown in FIG. 2, a germline filter 200 may be applied to the sequence reads. Some (e.g., less than all) or all steps shown in FIG. 2 may be performed in any combination and in any order. Samples collected over the course of a subject's treatment (e.g., samples collected at time T₀ and at time T₁) may have differing levels of tumor shedding and allele imbalance, meaning that variant classification at step 107/108 may be prone to assign differing somatic classifications for the same variant in the same subject. Since the aim of molecular response is to track the somatic variants over the course of treatment, a classification discrepancy may be automatically resolved to properly remove germline variants from consideration by reclassifying variants. For example, a variant may be classified as somatic at time T₀ and germline at time T₁. For example, a variant may be classified as germline at time T₀ and somatic at time T₁. For example, a variant may be classified as germline at time T₀ and not classified at time T₁. For example, a variant may be classified as somatic at time T₀ and not classified at time T₁. The germline filter 200 is configured to resolve such discrepancies and reassign variant classification.

As shown in FIG. 2, at step 201, a determination may be made for at least one variant in the sequence reads as to whether the variant is a deleterious variant (e.g., a frameshift or nonsense mutation) in a tumor suppressing gene (TSG). For example, the variant may be compared to a database of known TSG's. If the variant is a deleterious variant in a TSG, the variant may be classified as somatic, regardless of the classification result at step 107/108 (e.g., the classification will be changed from germline to somatic).

If the variant is not a deleterious variant in a TSG, the germline filter 200 may determine the maximum MAF of variants present in a sample and the maximum fraction of diploid genes for at least one variant in the sample at step 202. If, at step 203, the maximum fraction of diploid genes for a variant (in one of the at least two time points) indicates that the variant is somatic and the MAF for the variant (in one of the at least two time points) does not increase the maximum MAF, the variant may be classified as somatic, regardless of the classification result at step 107/108 (e.g., the classification will be changed from germline to somatic). If, at step 203, the maximum fraction of diploid genes for a variant (in one of the at least two time points) indicates that the variant is germline and the MAF for the variant (in one of the at least two time points) would increase the maximum MAF, the variant may be classified as germline, regardless of the classification result at step 107/108 (e.g., the classification will be changed from somatic to germline).

If, at step 203, the maximum fraction of diploid genes for the variant indicates that the variant is somatic and the MAF for the variant would increase the maximum MAF—or—if the maximum fraction of diploid genes for the variant indicates that the variant is germline and the MAF for the variant would not increase the maximum MAF, the germline filter 200, at step 204, may determine if the variant is classified as somatic in another patient sample at less than a threshold percentage (in one of the at least two time points). The threshold percentage may be at least about 1%, 2%, 3%, 4%, 5%, 6%, 7%, 8%, or 9%. If the variant is classified as somatic in another patient sample at less than the threshold percentage the variant may be classified as somatic, regardless of the classification result at step 107/108 (e.g., the classification will be changed from germline to somatic).

If, at step 204, the variant is not classified as somatic in another patient sample at <5%, the germline filter 200 may, at step 205, determine if the MAF for a variant (in one of the at least two time points) is larger than another MAF in the sample. For example, the germline filter 200 may determine if the MAF for the variant is at least about two times greater, three times greater, four times greater, five times greater, six times greater, seven times greater, eight times greater, nine times greater, or at least 10 times greater than one or more other MAFs in the same sample. The one or more other MAFs in the sample may be, for example, the next highest somatic MAF to the max MAF in the sample. If the MAF for the variant is larger than another MAF in the sample the variant may be classified as germline, regardless of the classification result at step 107/108 (e.g., the classification will be changed from somatic to germline).

The germline filter 200 may, at step 205, determine if the MAF for a variant (in one of the at least two time points) is larger than another MAF in another sample. For example, the germline filter 200 may determine if the MAF for the variant is at least about two times greater, three times greater, four times greater, five times greater, six times greater, seven times greater, eight times greater, nine times greater, or at least 10 times greater than one or more other MAFs in another sample. The one or more other MAFs in another sample may be, for example, the max MAF of the other sample. If the MAF for the variant is larger than another MAF in another sample the variant may be classified as germline, regardless of the classification result at step 107/108 (e.g., the classification will be changed from somatic to germline).

If, at step 205, the MAF for the variant is neither larger than another MAF in the sample nor larger than another MAF in another sample, the germline filter 200 may classify the variant as germline, regardless of the classification result at step 107/108 (e.g., the classification will be changed from somatic to germline).

Those variants classified as germline may be excluded from further analysis, including for example, MAF determination and/or MR scoring. In some embodiments, variants are classified as CHIP variants when those variants are classified as CHIP in at least one patient sample.

ii. CHIP Filter

cfDNA can comprise an aggregate of cfDNA from any cell types including tumor, blood cell and the like. Clonal hematopoiesis of intermediate potential mutation (CHIP) may even be present in cfDNA. Common approaches for CHIP filtering leverages recurrent CHIP genes or hotspots curated by large public or internal cohort studies.

However, these approaches do not address challenges in identifying random CHIP mutations in a plasma only approach. Residual unfiltered CHIP variants would bias the fractional change towards 1 (unchanged) and thus yield inaccurate subsequent molecular response prediction. To filter private CHIP variants (e.g., a variant that is CHIP but has not been documented ever or not often in previous databases of known CHIP variants), mutation measurement between two timepoints can be used to cluster variants of similar fractional change. When a patient receive treatment, progression or response will result in fractional somatic mutation while CHIP variant will remain stable. By clustering mutations into clones, random CHIP variants can be found in clones with enrichment of known CHIP list or in clones with stable fractional difference.

Accordingly, provided herein is an improvement in CHIP filtering that leverages the observations between two timepoints (T₀ and T₁) to cluster genomic mutations in clones with differing fractional change. CHIP filtering may group/cluster events into clones to estimate % clone load change. The clustering procedure may start with each single event and then merge utilizing a novel clustering heuristic. Once the % clone load change is determined using all the events, each clone can be inspected based on composition of variants and % clone load change to determine if the variant is a CHIP clone.

In one embodiment, the genomic mutations/variants are clustered utilizing a novel agglomerative hierarchical clustering heuristic. The heuristic quantifies the statistical dissimilarity between mutations/variants and clusters via a custom dissimilarity metric. A tunable stopping rule is utilized which continues agglomeration until a minimum (or maximum, depending upon the metric) allowable dissimilarity threshold is met. In one embodiment, the custom dissimilarity metric is a modification of the Bhattacharyya distance such that a numerical integration is performed with respect to the product (not subjected to a square root) of the scaled likelihoods of the mutations/variants and/or clusters that are under consideration to be merged at a given step of the clustering heuristic. The likelihoods are scaled to numerically integrate to 1 over the support of the integration. For SNVs and indels, the likelihood is calculated with respect to a Beta-Binomial model approximation of the observed count data that informs the MAF determination for the variants being clustered. The dispersion of the Beta-Binomial model is set via a tunable parameter. For CNVs, the likelihood is calculated with respect to a Gaussian model approximation of the observed fold change estimates of the mutations of interest, with the variability of Gaussian model also set via a tunable parameter. The agglomeration of mutations is conducted in a novel fashion such that, in some instances, clustering is performed via a tiered approach, in which a first set of mutations is clustered until the stopping rule is met and then a second set of mutations is introduced and further agglomerative steps are possibly performed according to the same dissimilarity metric and stopping rule. In some circumstances, a third set of mutations is introduced in a similar manner following the application of the clustering heuristic to the second set of mutations.

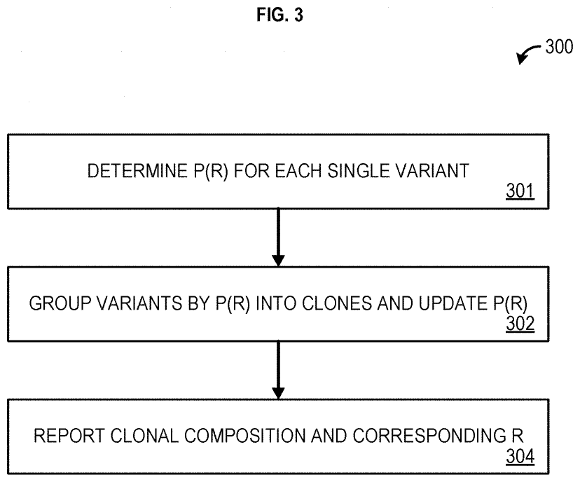

In an embodiment, shown in FIG. 3, a CHIP filter 300 may estimate, at step 301, a scaled likelihood function Pᵢ(Rᵢ) for each mutation/variant in a sample, where i=1, . . . , Iₘᵥ is the index for each unique qualifying mutation/variants observed across the two time points for a given sample, assuming a total of Iₘᵥ qualifying mutation/variants are observed. For ease of presentation, we denote the number of observed mutation/variant counts at time point 1 for the i^{th} mutation/variant as m_{i:1}^{obs}, and the total number of counts at the genomic location and at time point 1 as n_{i:1}^{obs}. Similarly, define m_{i:2}^{obs} and n_{i:2}^{obs}, but for time point 2. Define ν_{i:1}^{true} and ν_{i:2}^{true} as the true mutation/variant allele fractions as time points 1 and 2, respectively. The heuristic is designed to estimate Rᵢ=ν_{i:2}^{true}/ν_{i:1}^{true} and then to cluster together mutations/variants with Rᵢ values that can be plausibly considered to be identical. One embodiment of the heuristic is as follows:

| | True Variant | Unrestricted | Model Restricted |
|---|---|---|---|
| | Allele | Estimates of Variant | Estimates of Variant |
| Time | Fraction | Allele Fraction | Allele Fraction |
| T₀ | υ_{i:1}^{true} | v_{i:1}^{obs} = m_{i:1}^{obs}/n_{i:1}^{obs} | υ_{i:1} = v_{i:1}^{obs} |
| T₁ | υ_{i:2}^{true} | v_{i:2}^{obs} = m_{i:2}^{obs}/n_{i:2}^{obs} | υ_{i:2} = rᵢv_{i:1}^{obs} |

Pᵢ(Rᵢ) may be determined as:

Pᵢ(Rᵢ=rᵢ)=cᵢf_{i:1}(m_{i:1}^{obs},n_{i:1}^{obs})f_{i:2}(m_{i:2}^{obs},n_{i:2}^{obs},m_{i:1}^{obs},n_{i:1}^{obs},rᵢ)

where

f_{i:1}(m_{i:1}^{obs},n_{i:1}^{obs})˜Binomial(x=m_{i:1}^{obs},N=n_{i:1}^{obs},p=v_{i:1}^{obs})

and

f_{i:2}(m_{i:2}^{obs},n_{i:2}^{obs},m_{i:1}^{obs},n_{i:1}^{obs},rᵢ)˜Binomial(x=m_{i:2}^{obs},N=n_{i:2}^{obs},p=rᵢv_{i:1}^{obs})

and

cᵢ is calculated such that the numeric integration of Pᵢ(Rᵢ=rᵢ) across a support of candidate rᵢ values is equal to 1. This example embodiment assumes that the data is not over-dispersed with respect to the Binomial model and corresponds to a special case of the more general class of Beta-Binomial models.

Approximate confidence intervals can be calculated for Rᵢ in a variety of ways, including via a highest density interval like approach in which the scaled likelihood for Pᵢ(Rᵢ=rᵢ) is considered to be an approximate posterior density estimate of Rᵢ assuming an improper prior distribution for the Rᵢ values.

At step 302, the set of mutations/variants may be pairwise agglomerated according to Pᵢ(Rᵢ). For all possible pairings {i′, i*:≠i*; i′, i*=1, 2, . . . , Iₘᵥ} the dissimilarity measure, D(i′, i*), between P_{i′}(R_{i′}) and P_{i*}(R_{i*}) is calculated using a modified Bhattacharyya distance. Larger values of D(i′, i*) indicate that the mutation pair {i′,i*} are more likely to be realizations from the same underlying fractional change distribution. Accordingly, a pair of mutations/variants with the greatest value of D (⋅,⋅) may be merged into a single clone and Pᵢ(Rᵢ) for that clone may be updated. Pairwise agglomerations may continue until stopping criteria are satisfied or all mutations/variants have agglomerated to a single clone. The threshold may be and/or include values ranging from about 0.0005 to 0.005.

At step 303, the number of clones and associated fractional change between timepoints may be reported with a confidence interval. Clones having a fractional change between the first and second time points at or above a predetermined threshold value may be identified. If multiple clones are identified, clones with a fractional change close to 1 and/or clones with specific known CHIP variants may be classified as potential CHIP variants. CHIP variants may be excluded from further analysis. In some embodiments, variants may be classified as CHIP variants when those variants are classified as CHIP in at least one patient sample.

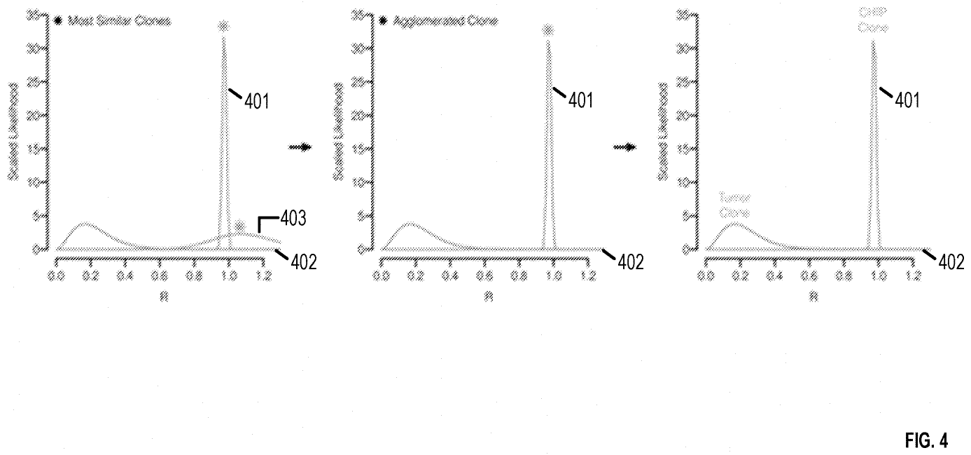

FIG. 4 shows an example application of the CHIP filter 300. FIG. 4 corresponds to an example of an agglomeration procedure. In the example, there are three qualified mutants identified. The left most panel of FIG. 4 displays the scaled likelihood functions for each mutant (y-axis) over the support of R (x-axis). Suppose the mutant corresponding to a first likelihood (line 403) of the scaled likelihood functions for each mutant is a known CHIP mutation. Mutants in the left panel with the most similarity are annotated with stars. The middle panel displays the resulting agglomerated likelihood from the merging of the first likelihood (line 403) and a second likelihood (line 401) of the scaled likelihood functions for clones in the left panel. A third likelihood (line 402) of the scaled likelihood functions for each clone from the left panel has a likelihood function that is unaltered by the agglomeration. The right panel displays the final clonality. Since the composition of the second likelihood (line 401) clone is 50% CHIP, the second likelihood (line 401) clone may be identified as putatively CHIP. This would result in the final value of R being defined solely by the third likelihood (line 402) clone.

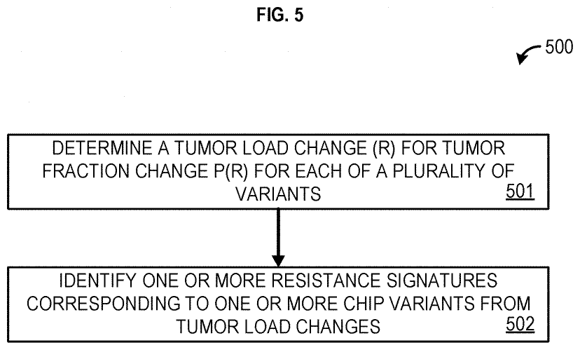

In an embodiment, shown in FIG. 5 is a flow chart that schematically depicts exemplary method steps of identifying clonal hematopoietic variants in a subject having cancer according to some embodiments. As shown, method 500 includes determining a tumor load change (R) for tumor fraction change P(R) for each of a plurality of variants from sequence information generated from targeted nucleic acids associated with one or more cancer types in samples obtained from the subject at first and second time points to produce a set of tumor load changes (step 501). In addition, method 500 also includes identifying one or more resistance signatures corresponding to one or more clonal hematopoietic variants from the set of tumor load changes (step 502).

f. MR Score

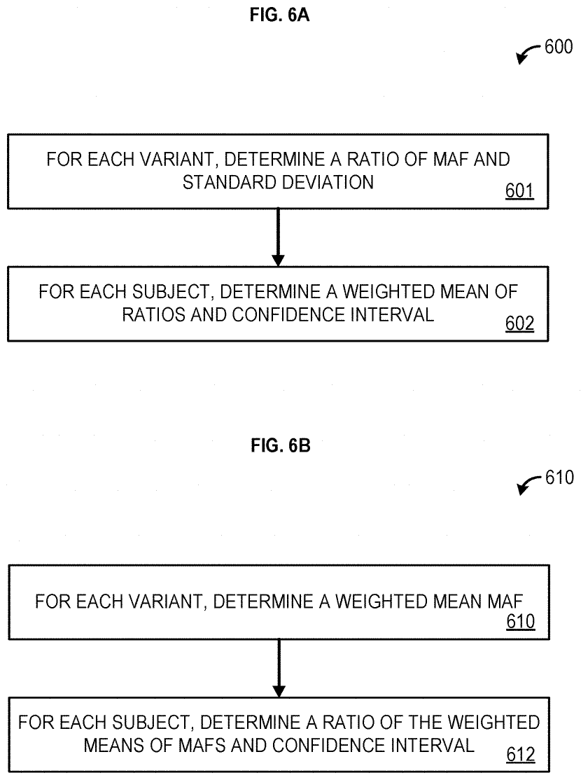

Returning to FIG. 1, the method 100 may proceed to determine an MR score at step 110. In an embodiment, the MR score may be determined using MAF values associated with somatic variants remaining after variant filtering at step 109. In an embodiment, MAF values of all the somatic variants may be used. In an embodiment, MAF values of less than all the somatic variants may be used. As described at step 105/106, MAFs may be determined for a plurality of somatic variants from sequence reads generated from targeted nucleic acids associated with one or more cancer types in samples obtained from the subject at T₀ (e.g., pre-treatment) and T₁ (e.g., on-treatment) to produce sets of first and second MAFs for somatic variants in the plurality of somatic variants. An MR score can be expressed as a fraction or as a percentage. As shown in FIG. 6A, an MR score may be determined according to a method 600. The method 600 may comprise determining a ratio of the first MAFs and second MAFs for somatic variants in the plurality of somatic variants to produce a set of MAF ratios and a corresponding standard deviation for an MAF ratio in the set of MAF ratios at step 601. In some embodiments, the standard deviation can be utilized as a criterion for reporting the MR score. For example, the standard deviation of the MR score, based on the individual standard deviations of at least one variant, can be used to determine a confidence interval and a subsequent cutoff for sample evaluability. In some embodiments, the cutoff can be at least 0.1, 0.15, 0.2, 0.3, 0.4 or 0.5. At step 602, for a subject, a weighted mean of the MAF ratios may be determined using the formula:

$$\frac{\sum ( weight * ratio )}{\sum weights}$$

where weight is 1/range\hat{ }2 for a given somatic variant in the plurality of somatic variants, where range is a difference between values of the first and second MAFs for a given somatic variant in the plurality of somatic variants, and ratio is a given MAF ratio in the set of MAF ratios. A confidence interval may be determined using the formula: weighted mean of the MAF ratios+/−\sqrt{ratio variance},
where ratio variance is

$$\frac{1}{\sum weights} .$$

In an embodiment, in addition to, or as an alternative to, the weighted mean of MAF ratios as an MR score, a method is disclosed that clusters variants based on MAF ratios, calculates an aggregate MAF ratio for the cluster, and then uses as the MR score either a single selected cluster ratio or the weighted mean of the cluster ratios. The clustering may be performed by combining pairs of variants with overlapping MAF ratio distributions, or other clustering methods. The single selected cluster may be that which contains a known cancer driver variant, or absence of known clonal hematopoiesis variants. Cluster weights may also depend on the presence of a known cancer driver variant or the maximum VAF or number of variants in the cluster.

As shown in FIG. 6B, an MR score may be determined according to a method 610. The method 610 may comprise determining a weighted mean of the first MAFs and a weighted mean of the second MAFs for a somatic variant in the plurality of somatic variants and a corresponding standard deviation for a weighted MAF ratio at step 601. In some embodiments, the standard deviation can be utilized as a criterion for reporting the MR score. For example, the standard deviation of the MR score, based on the individual standard deviations of at least one variant, can be used to determine a confidence interval and a subsequent cutoff for sample evaluability. In some embodiments, the cutoff can be at least 0.1, 0.15, 0.2, 0.3, 0.4 or 0.5. At step 612, for a subject, a ratio of the weighted means of the MAFs may be determined. A confidence interval as the variance of the ration. For example, confident interval may be determined using the formula:

R=A/B: var(R)˜=var(B)/A\hat{ }2+var(A)*B\hat{ }2/A\hat{ }4,

where A and B are the weighted mean MAF at timepoint 1 and timepoint 2 respectively.

Clusters may be weighted based on the strength of evidence. For example, the max-VAF may indicate which is the primary clone, the number of non-CHIP variants may weight the cluster with the stronger signal; the driver weight may increase weight or select the cluster that contains the driver for that particular cancer type or molecular subtype. The weighting applied may be, for example, applying a greater weight to variants known to be drivers in the specific cancer type or molecular subtype. In an embodiment, weights may be based on max-VAF (either sample), number of non-CHIP variants, and/or driver weight (tumor-type-specific; defined in configuration file). In another embodiment, the weighting applied may be, for example, weighting somatic variants equally.

In an embodiment, classification as a molecular responder or a molecular non-responder may depend on the variant VAFs and variant weights. For example, if the MR score is the ratio of mean VAFs, then the higher VAF (i.e., more clonal variant) is likely to dominate. If the MR score uses variant weights, then the variant with the higher weight (e.g., driver variant) might dominate.

The resulting weighted mean of the MAF ratios as described in FIG. 6A or the ratio of the weighted means of the MAFs as described in FIG. 6B is the MR score for the subject. Such an MR score incorporates the variance of MAF into the molecular response calculation. This ensures molecular response scores include accurate variance, which contributes to drawing a correct conclusion from the molecular response. The MR score may be viewed as a “numerically stable” ratio of mean MAFs, which appropriately weights changes in MAF based on the precision in the MAF, and which is not susceptible to overconfident and incorrect results when MAFs are fluctuating near the limit of detection (LOD). The MR score may be compared to a threshold to determine if the subject is responding to treatment or not responding to treatment. The threshold may be and/or include, for example, from about 25% to about 75%. In some embodiments, weighting could be either based on VAF precision (e.g. position, hotspot region, coverage depth and the like) or prior knowledge of importance of that variant to the tumor (e.g. known driver or resistance mutation, or variant of uncertain (or unknown) significance).

To provide a simple example to illustrate aspects of the problem that the MR scoring methods presented herein address, consider a subject with one variant detected, with an MAF of 0.3% at baseline (T₀), and an MAF 0.1% on treatment (T₁), and a coverage at that variant position of 3000 molecules. Using Dre-existing methods, the molecular response score would be:

$$\frac{0.1 \%}{0.3 \%} = 33 \% .$$

For a cutoff to define “molecular responder” vs “molecular non-responder” of 50%, this subject would be a “molecular responder.” However, propagating the variance according to the methods described herein results in a molecular response score with an expected value of ˜30-40%, but a 95% confidence interval of 0-120%. Therefore, for this subject, the molecular response should be considered not evaluable, because it cannot be confidently assessed whether the MR score is truly below or above the 50% cutoff.

To provide a simple example to illustrate aspects of the problem that the MR scoring methods presented herein address, consider a subject with two variants (a and b) detected, with MAFs of a=0.1% and b=8.0% at baseline (T₀), and MAFs a=0.3% and b=2.0% on treatment (T₁). Using pre-existing methods taking the mean of ratios, the molecular response score would be: mean

$$( \frac{0.3 \%}{0.1 \%} , \frac{8.0 \%}{2. \%} ) = 163 \% .$$

For a cutoff to define “molecular responder” vs “molecular non-responder” of 50%, this subject would be a “molecular non-responder.” However, using the ratio of means according to the methods described herein the molecular response score would be

$$\frac{mean ( 0.3 \% , 2. \% )}{mean ( 0.1 \% , 8. \% )} = 28 \% .$$

Therefore, for this subject, the molecular response should be considered “molecular responder.”

To provide a simple example to illustrate aspects of the problem that the MR scoring methods presented herein address, consider a subject with two variants (a and b) detected, with MAFs of a=0.3% and b=0.0% at baseline (T₀), and MAFs a=0.0% and b =0.3% on treatment (T₁). Using pre-existing methods to only evaluate variants above 0.3% at baseline, the molecular response score would be:

$$\frac{0. \%}{0.3 \%} = 0 \% .$$

For a cutoff to define “molecular responder” vs “molecular non-responder” of 50%, this subject would be a “molecular responder.” However, including variants that arise on-treatment, the molecular response score would be

$$\frac{mean ( 0.3 \% , 0. \% )}{mean ( 0. \% , 0.3 \% )} = 100 \% .$$

Therefore, for this subject, the molecular response should be considered “molecular non-responder.”

The method 100 may include administering one or more therapies to the subject based upon at least the molecular response score. Exemplary therapies are disclosed further herein. In some embodiments, the method 100 includes comparing the molecular response score for the subject having the cancer to a predetermined cutoff point to identify that the subject is a likely responder to one or more therapies (e.g., immunotherapies or the like) for the cancer when the molecular response score is below the predetermined cutoff point or that the subject is a likely non-responder to the one or more therapies for the cancer when the molecular response score is at or above the predetermined cutoff point. In some embodiments, the method 100 includes administering one or more therapies for the cancer to the subject in view of the molecular response score. In some embodiments, the method 100 includes discontinuing administering one or more therapies for the cancer to the subject in view of the molecular response score. In some embodiments, the method 100 includes using the molecular response score as a prognostic biomarker and/or a predictive biomarker for the subject.

In other exemplary embodiments, variance is incorporated into the molecular response calculation through simulation or sampling from the variance distribution of at least one variant to calculate the molecular response variance. As further disclosed herein, some applications include weighting variants based on their importance in the tumor or likelihood of tumor vs clonal hematopoeisis. Some embodiments involve integrating multiple genomic data sources to estimate tumor fraction (instead of just relying on variant (e.g., SNV, Indel and Fusion) VAFs), coverage (e.g., copy number), off-target coverage, and/or methylation, among other genomic data sources.

In some embodiments, the methods include using one or more additional genomic data sources to determine the molecular response score for the subject having the cancer. In some embodiments, the additional genomic data sources comprise one or more of: a coverage, an off-target coverage, an epigenetic signature, tumor mutational burden and/or a microsatellite instability score. For a data source, there can be a calculation of tumor fraction based on that data source, and the calculated tumor fraction may be combined across data sources (for example using a weighted mean, incorporating the confidence of a data source in the tumor fraction for that particular sample), and then the overall tumor fraction estimate in a sample may be combined to calculate an overall molecular response. In some embodiments, the epigenetic signature comprises a cfNA fragment length, position, and/or endpoint density distribution. In some embodiments, the epigenetic signature comprises an epigenetic state or status exhibited by one or more epigenetic loci in a given targeted genomic region. In some embodiments, the epigenetic state or status comprises a presence or absence of methylation, hydroxymethylation, acetylation, ubiquitylation, phosphorylation, sumoylation, ribosylation, citrullination, and/or a histone post-translational modification or other histone variation.

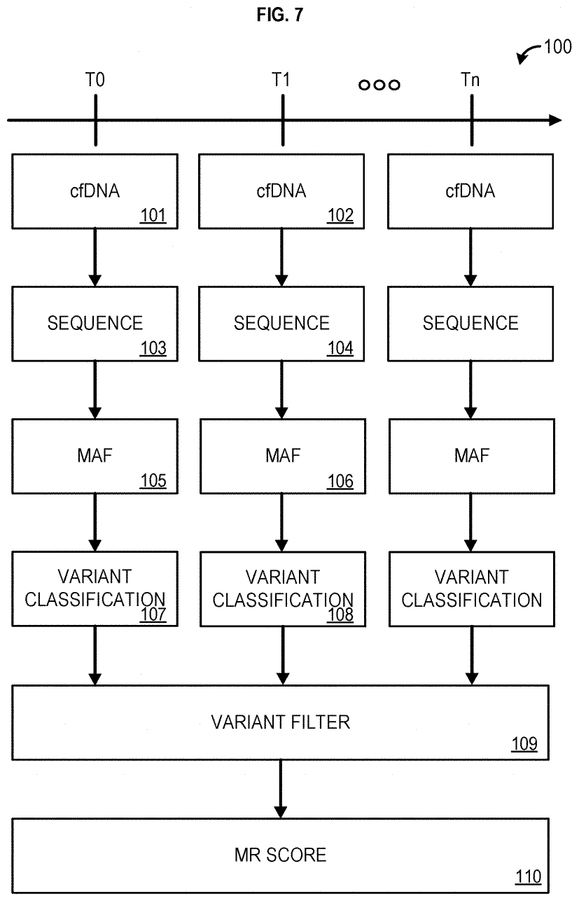

While the present methods are described in the context of FIG. 1 and first time T₀ and second time T₁, it is to be understood that more than two time points are contemplated, for example for longitudinal monitoring. As shown in FIG. 7, at the first time T₀, baseline cfDNA may be obtained from one or more baseline samples obtained from one or more subjects prior to treatment and at a second time T₁, or any subsequent time T₀, on-treatment cfDNA may be obtained from one or more on-treatment samples obtained from one or more subjects after treatment. Time T₁ can be any amount of time after time T₀, for example, any time between and including 1-24 hours, 1-180 days, 1-12 weeks, 1-25 weeks, 1-30 weeks and the like. Moreover, the method 100 may be applied to any combinations of times T₀, T₁, . . . , Tₙ. For example, samples may be obtained at time T₁ and at a time T₂, wherein samples taken at both times are on-treatment samples. In another example, samples may be obtained at time T₁ and at a time T₂, wherein a sample taken at time T₁ represents an on-treatment sample and a sample taken at time T₂, represents an off-treatment sample.

In an embodiment, a dosage of a therapy being administered to the subject may be adjusted based on the molecular response score. For example, the molecular response score may indicate that the subject is not responding to a first treatment and the dosage of the first treatment may be increased in response. In an embodiment, an alternative therapy may be identified based on the molecular response score. For example, the molecular response score may indicate that the subject is not responding to a first treatment and the subject may then be placed on a second treatment in place of, or in addition to, the first treatment. In an embodiment, a molecular response score may be determined for subjects in a clinical trial, wherein molecular response scores may be determined for subjects receiving a placebo and for subjects receiving treatment. The molecular response scores of the two categories of subjects may be compared to assess the treatment.

In another example, placebo and treatment may be generalized to two arms of a clinical trial comparing different combinations of drugs. The threshold or cutoff may be specific to the use case: the use case may require clearance (MR=0) or the use case may require a certain level of decrease or increase of ctDNA level.

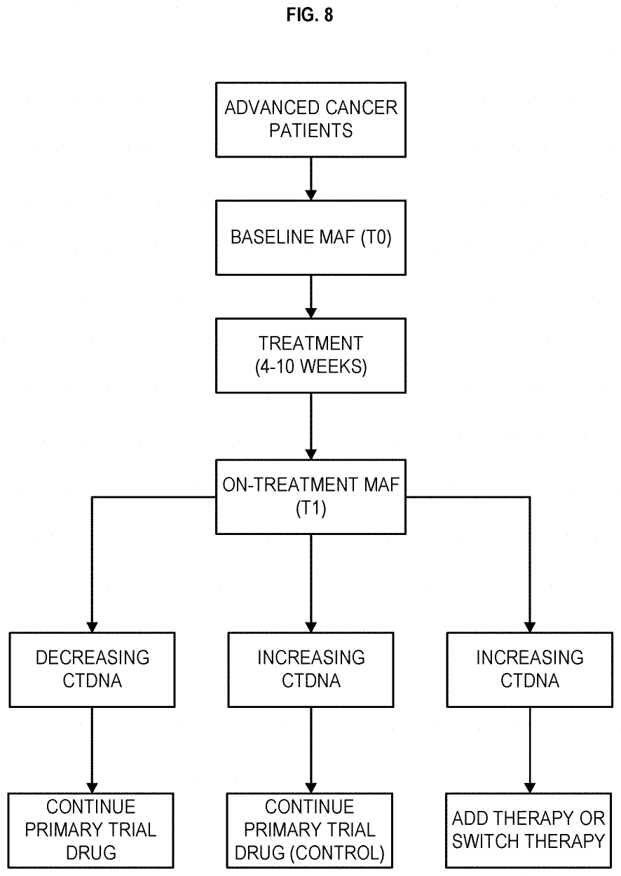

FIG. 8 shows an example practical application of the molecular response score for patient stratification. Advanced cancer patients may have a baseline MAF determined at time T₀, prior to treatment. After 4-10 weeks of treatment, the advanced cancer patients may have an on-treatment MAF determined at time T₁. The resulting molecular response score may indicate that ctDNA in a patient is decreasing, in which case the patient should continue to be treated with the primary trial drug. The resulting molecular response score may indicate that ctDNA in a patient is increasing, in which case the patient should continue to be treated with the primary trial drug (or with placebo) if the patient is in a control group. Otherwise, if ctDNA in a patient is increasing, the patient should have one or more therapies added to their treatment regime, a therapy changed, or a dose of the primary trial drug changed.

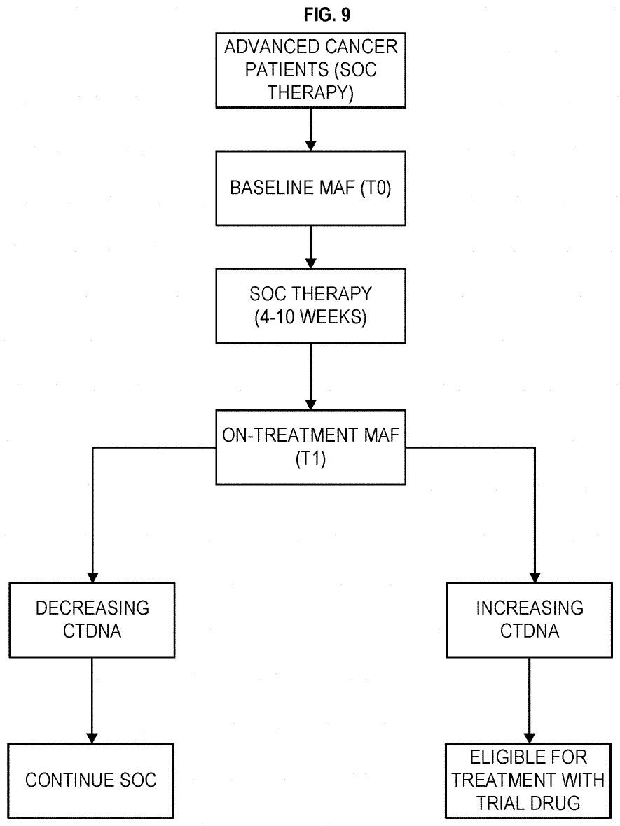

FIG. 9 shows an example practical application of the molecular response score for clinical trial enrichment. Advanced cancer patients eligible for standard of care (SOC) treatment may have a baseline MAF determined at time T₀, prior to SOC treatment. After 4-10 weeks of SOC treatment, the advanced cancer patients may have an on-treatment MAF determined at time T₁. The resulting molecular response score may indicate that ctDNA in a patient is decreasing, in which case the patient should continue to be treated with the SOC treatment. The resulting molecular response score may indicate that ctDNA in a patient is increasing, in which case the patient may be determined eligible for treatment with a clinical trial drug.

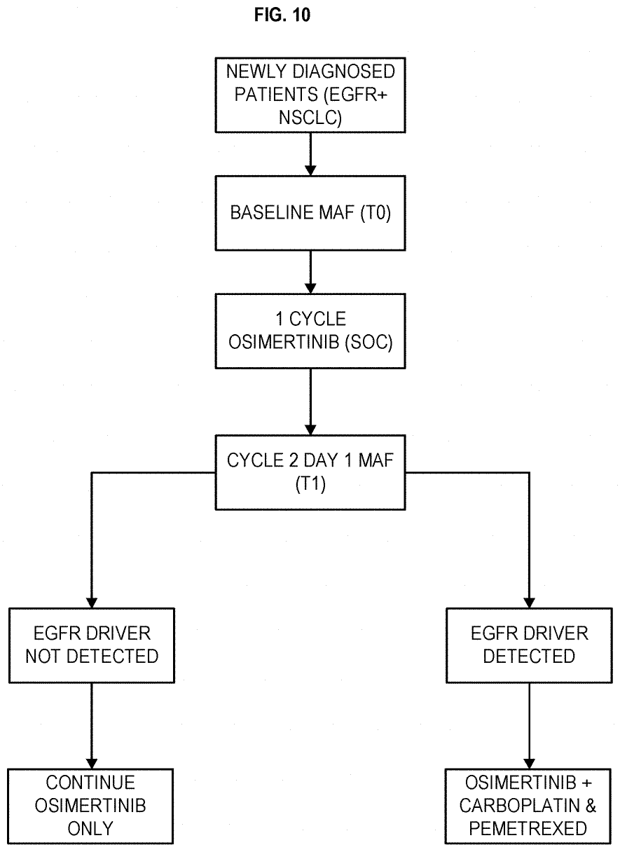

FIG. 10 shows an example practical application of the molecular response score for prospective patient stratification and escalation for a MSKCC trial of osimertinib+/−chemotherapy in patients with EGFR-positive non-small cell lung cancer (NSCLC). Newly diagnosed patients with EGFR-positive NSCLC may have a baseline MAF determined at time T₀, prior to with osimertinib. After 1 cycle of osimertinib, the patient may have an on-treatment MAF determined at day 1 of cycle 2 of osimertinib. The resulting molecular response score, based only on the EGFR driver, may indicate that the EGFR driver is not detected, in which case the patient should continue to be treated with osimertinib only. The resulting molecular response score, based only on the EGFR driver, may indicate that the EGFR driver is detected, in which case the patient should continue to be treated with osimertinib, carboplatin, and pemetrexed.

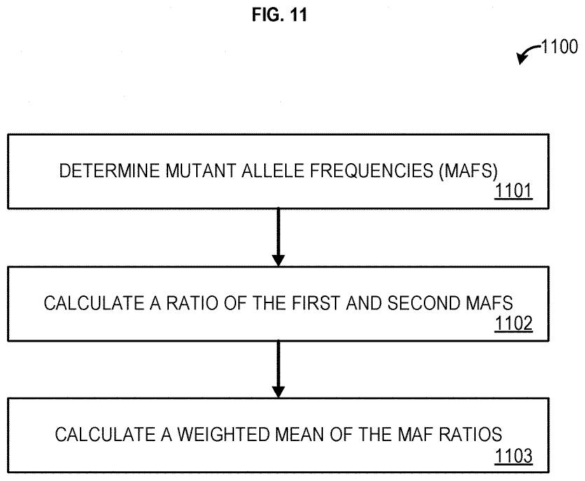

Aspects of these methods are further illustrated in FIG. 11. As shown, method 1100 includes determining mutant allele frequencies (MAFs) for a plurality of variants from sequence information generated from targeted nucleic acids associated with one or more cancer types in samples obtained from the subject at first (e.g., pre-treatment) and second (e.g., on-treatment) time points to produce sets of first and second MAFs for a variant in the plurality of variants (step 1101). Method 1100 also includes calculating a ratio of the first and second MAFs for a variant in the plurality of variants to produce a set of MAF ratios and a corresponding standard deviation for a MAF ratio in the set of MAF ratios (step 1102). In addition, method 1100 also includes calculating a weighted mean of the MAF ratios (step 1103) and a confidence interval to determine the molecular response score for the subject having the cancer.

In some embodiments, method 1100 includes comparing the molecular response score for the subject having the cancer to a predetermined cutoff point to identify that the subject is a likely responder to one or more therapies (e.g., immunotherapies or the like) for the cancer when the molecular response score is below the predetermined cutoff point or that the subject is a likely non-responder to the one or more therapies for the cancer when the molecular response score is at or above the predetermined cutoff point. In some embodiments, method 1100 includes administering one or more therapies for the cancer to the subject in view of the molecular response score. In some embodiments, method 1100 includes discontinuing administering one or more therapies for the cancer to the subject in view of the molecular response score. In some embodiments, method 1100 includes using the molecular response score as a prognostic biomarker and/or a predictive biomarker for the subject.

In some embodiments, method 1100 includes using a molecule count to calculate the standard deviation for a MAF ratio in the set of MAF ratios. In some embodiments, method 1100 includes propagating a variance through a MAF ratio in the set of MAF ratios. In some embodiments, method 1100 includes excluding one or more germline and/or clonal hematopoietic variants when determining the mutant allele frequencies (MAFs) for the plurality of variants. Examples of methods of excluding germline and CHIP variants are described further herein. In some embodiments, method 1100 includes excluding one or more somatic variants having MAFs that are less than about 0.1%, 0.2%, 0.3%, 0.4%, 0.5%, 0.6%, 0.7%, 0.8%, or 0.9% at the first and/or second time points. In some embodiments, the method comprises excluding one or more somatic variants less than 5, 10, 15, 20, 25 or 30 mutant molecule counts at the first and/or second time points. In some embodiments, the method comprises excluding one or more somatic variants having a coverage less than 300, 400, 500, 600, 700, 800, 900 or 1000 at the first and/or second time points. In some of these embodiments, the first time point comprises a pre-treatment time point and wherein the second time point comprises an on- or post-treatment time point.

In some embodiments, the methods disclosed herein include generating the sequence information from nucleic acid molecules obtained from one or more tissues or cells in the sample. In some embodiments, the methods disclosed herein include generating the sequence information from cell-free nucleic acids (cfNAs) in the samples obtained from the subject. In some embodiments, the cfNAs comprise circulating tumor DNA (ctDNA).

In some embodiments, the ratio comprises the second MAF to the first MAF for a variant in the plurality of variants. In some embodiments, method 1100 includes calculating the weighted mean of the MAF ratios using the formula:

sum[weight*ratio]/sum[weights],

where weight is 1/range² for a given variant in the plurality of variants, where range is a difference between values of the first and second MAFs for a given variant in the plurality of variants, and ratio is a given MAF ratio in the set of MAF ratios. In some embodiments, method 1100 includes calculating the confidence interval using the formula:
weighted mean of the MAF ratios+/−sqrt[ratio variance],
where ratio variance is 1/sum[weights].

In some embodiments, the variants comprise one or more single-nucleotide variants (SNV), insertion/deletion mutations (indels), gene amplifications, and/or gene fusions. In some embodiments, method 1100 includes using one or more additional genomic data sources to determine the molecular response score for the subject having the cancer. In some embodiments, the additional genomic data sources comprise one or more of: a coverage, an off-target coverage, an epigenetic signature, and/or a microsatellite instability score. In some embodiments, the epigenetic signature comprises a cfNA fragment length, position, and/or endpoint density distribution. In some embodiments, the epigenetic signature comprises an epigenetic state or status exhibited by one or more epigenetic loci in a given targeted genomic region. In some embodiments, the epigenetic state or status comprises a presence or absence of methylation, hydroxymethylation, acetylation, ubiquitylation, phosphorylation, sumoylation, ribosylation, citrullination, and/or a histone post-translational modification or other histone variation.

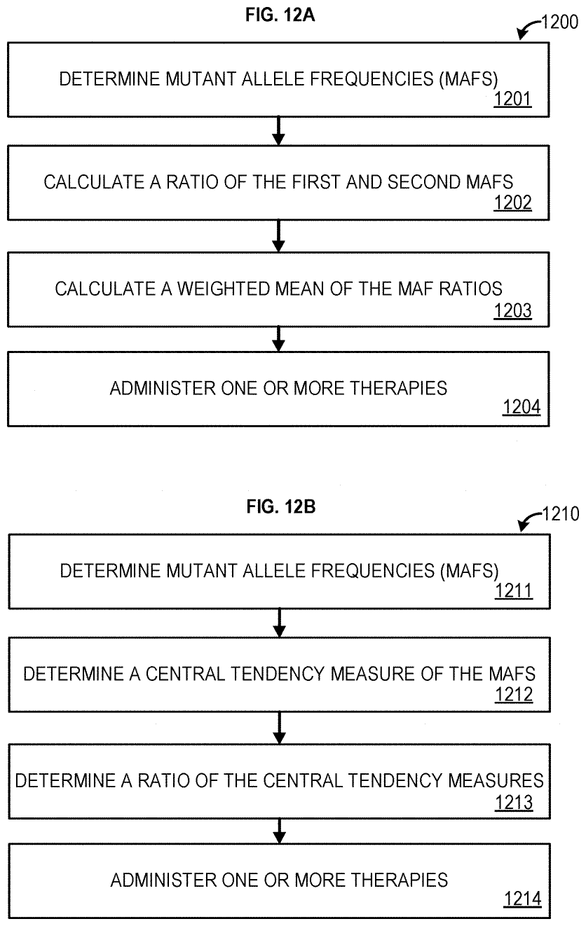

To further illustrate, FIG. 12A is a flow chart that schematically depicts an example method 1200. As shown, method 1200 includes determining mutant allele frequencies (MAFs) for a plurality of variants from sequence information generated from targeted nucleic acids associated with one or more cancer types in samples obtained from a subject at first and second time points to produce sets of first and second MAFs for a variant in the plurality of variants (step 1201). Method 1200 also includes calculating a ratio of the first and second MAFs for a variant in the plurality of variants to produce a set of MAF ratios and a corresponding standard deviation for a MAF ratio in the set of MAF ratios (step 1202) and calculating a weighted mean of the MAF ratios and a confidence interval to determine a molecular response score for the subject (step 1203). In some embodiments, the standard deviation can be utilized as an estimate of confidence interval. In some embodiments, the standard deviation can be utilized as a criteria for reporting the molecular response score. In addition, method 1200 also includes administering one or more therapies to the subject based upon at least the molecular response score (step 1204). Exemplary therapies are disclosed further herein.

FIG. 12B is a flow chart that schematically depicts an example method 1210. As shown, method 1210 includes determining mutant allele frequencies (MAFs) for a plurality of variants from sequence information generated from targeted nucleic acids associated with one or more cancer types in samples obtained from a subject at first and second time points to produce sets of first and second MAFs for a variant in the plurality of variants (step 1211). The method 1210 comprises determining a central tendency measure obtained from the MAFs of somatic variants considered for a time point (i.e., first time point and second time point) at step 1212. It is understood that the central tendency measure may be one of, although not limited to, a mean, median, or mode. The method 1210 comprises determining a ratio of the central tendency measure at the first time point to the central tendency measure at the second time point at step 1213. The method 1210 may comprise calculating a standard deviation of the central tendency ratio using the standard deviation of the MAFs considered. In some embodiments, the central tendency measure can be a mean or median. In some embodiments, the central tendency measure can be a mean. In some embodiments, the central tendency measure can be a median. In some embodiments, the method 1210 comprises determining a mean of the MAFs of somatic variants considered for each time point (i.e., first time point and second time point) at step 1212; calculating a ratio of the mean obtained at the first time point to the mean obtained at the second time point at step 1213 and calculating a standard deviation of the mean ratio using the standard deviation of each of the MAFs considered. In some embodiments, the molecular response score can be calculated from the ratio of the mean obtained at first time point to the mean obtained at second timepoint. In some embodiments, the method 1210 comprises determining a median of the MAFs of somatic variants considered for each time point (i.e., first time point and second time point) at step 1212; calculating a ratio of the median obtained at the first time point to the median obtained at the second time point at step 1213, and calculating a standard deviation of the median ratio using the standard deviation of each of the MAFs considered. In some embodiments, the molecular response score can be calculated from the ratio of the median obtained at first time point to the median obtained at second timepoint. In some embodiments, the standard deviation can be utilized as an estimate of confidence interval. In some embodiments, the standard deviation can be utilized as a criteria for reporting the molecular response score. In addition, method 1210 also includes administering one or more therapies to the subject based upon at least the molecular response score (step 1214). Exemplary therapies are disclosed further herein.

Typically, the methods of determining molecular response scores include filtering out CHIP variants. For example, molecular response is typically measured by allele frequency of genomic alternations (e.g., small variants between two time points) to represent tumor fractional change. Given that cfDNA signal is an aggregation of signal from essentially any cell types, including tumor, blood cell, and the like, numerous studies have shown the presence of clonal hematopoiesis of intermediate potential (CHIP) variants in cfDNA samples. Common approaches for CHIP filtering frequently leverage recurrent CHIP genes or hotspots curated by various data sources. However, it is yet a challenge to identify random CHIP mutations with a plasma only approach. Residual unfiltered CHIP variants typically bias the fractional change towards 1 (unchanged) and thus yield inaccurate molecular response prediction or scores. Accordingly, in some embodiments, the methods disclosed herein use a model to leverage the observations between two time points to cluster genomic mutations in clones with separate fractional change. To group mutations, these approaches typically leverage the variant allele count and total count for a variant from the two time points and build a probability density function for tumor fraction change R as P(R).

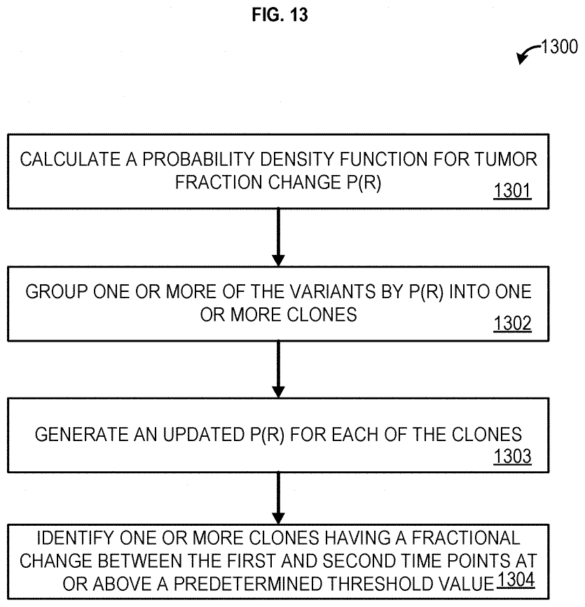

As a further illustration, FIG. 13 is a flow chart that schematically depicts exemplary method steps of identifying clonal hematopoietic variants in a subject having cancer according to some embodiments. As shown, method 1300 includes calculating a probability density function for tumor fraction change P(R) for a variant of a plurality of variants from sequence information generated from targeted nucleic acids associated with one or more cancer types in samples obtained from the subject at first and second time points (step 1301). As additionally shown, method 1300 also includes grouping one or more of the variants by P(R) into one or more clones (step 1302), generating an updated P(R) for a clone of the clones (step 1303), and identifying one or more clones having a fractional change between the first and second time points at or above a predetermined threshold value (step 1304).

In other aspect, this disclosure provides methods of identifying and excluding germline variants, or otherwise resolving somatic classification discrepancies when determining molecular response scores. For example, one problem is that samples collected over the course of a patient's treatment course typically have differing levels of tumor shedding and allele imbalance, meaning that a somatic variant caller of a given bioinformatics pipeline will sometimes arrive at differing somatic classifications for the same variant in the same patient. Since an aim of molecular response determinations is to track the somatic variants over the course of treatment, any classification discrepancies should be resolved to properly remove germline variants from consideration.

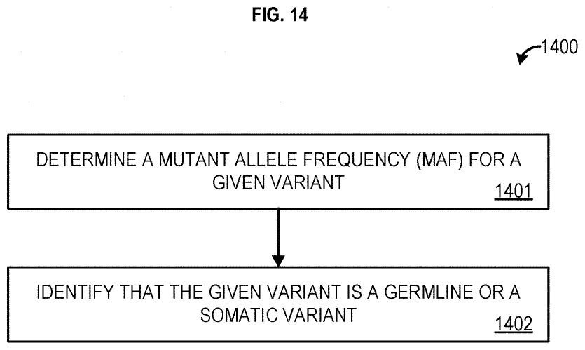

To illustrate, FIG. 14 is a flow chart that schematically depicts exemplary method steps of identifying variants in a subject having cancer according to some embodiments. As shown, method 1400 includes determining a mutant allele frequency (MAF) for a given variant from sequence information generated from targeted nucleic acids associated with one or more cancer types in a sample obtained from the subject (step 1401). The method 1400 may utilize the determined MAF for the given variant to identify the given variant as a germline or a somatic variant. In some embodiments, the method 1400 may utilize a baseline MAF and a subsequent on-treatment MAF for the given variant to classify, or change a previous classification of, the given variant as a germline or a somatic variant. The method 1400 may also include identifying that the given variant is a germline variant when the MAF of the given variant increases the max MAF of the sample (in one of the at least two time points) that comprises a maximum fraction of diploid genes (max frac_diploid) (i.e., least allele imbalance) and/or when the MAF of the given variant is at least about two times greater, three times greater, four times greater, five times greater, six times greater, seven times greater, eight times greater, nine times greater, or at least 10 times greater than one or more other MAFs (e.g., max MAF in a sample) determined from the sample obtained from the subject or another patient sample. In some embodiments, a given variant is classified as somatic when it does not raise the max MAF (e.g., compared to another MAF) of the sample in one of the at least two time points with max frac_diploid is somatic. In some embodiments, a given variant is classified as germline when it does raise the max MAF and the sample with max frac_diploid is germline. In some embodiments, method 1400 includes classifying a given variant as somatic when that variant is determined to be a deleterious variant (e.g., a frameshift or nonsense mutation) in a tumor suppressor gene (TSG). In some embodiments, a given variant is classified as somatic when it is seen at less than about 1%, 2%, 3%, 4%, 5%, 6%, 7%, 8%, or 9% in any given sample. In some embodiments, a given variant is classified as germline when the related discrepancy is not resolve by method 1400. In these embodiments, the variant is typically removed from further consideration when determining a given molecular response score. In some embodiments, variants are classified as CHIP variants when those variants are classified as CHIP in at least one patient sample.

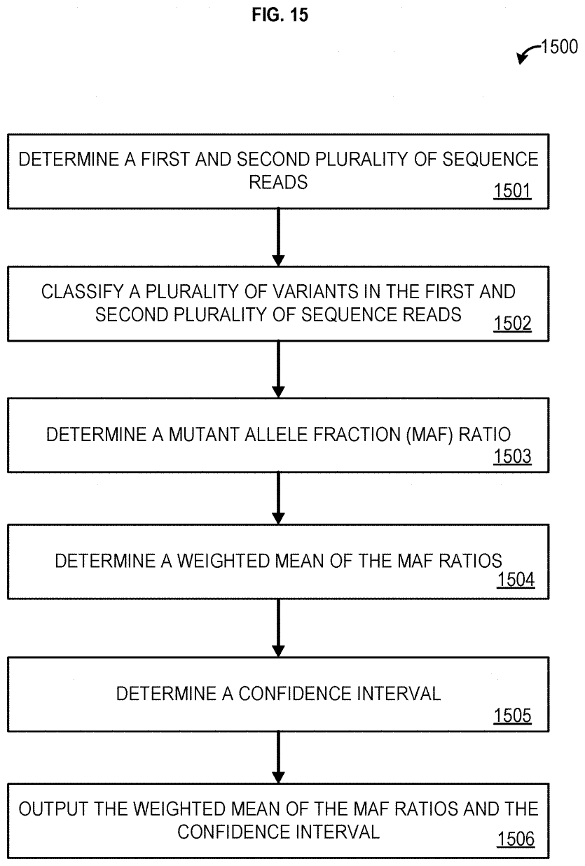

FIG. 15 is a flow chart that schematically depicts a method 1500 that includes determining a first plurality of sequence reads and a second plurality of sequence reads associated with a subject (step 1501). As additionally shown, method 1500 also includes classifying a plurality of variants in the first plurality of sequence reads and the second plurality of sequence reads as somatic or germline (step 1502), determining an MAF ratio (step 1503), determining a weighted mean of the MAF ratios (step 1504), determining a confidence interval associated with the weighted mean of the MAF ratios (step 1505), and outputting the weighted mean of the MAF ratios and the confidence interval (step 1506). It is understood that the first plurality of sequence reads may be determined before administering the therapy and the second plurality of sequence reads may be determined after administering the therapy. Classifying the plurality of variants in the first plurality of sequence reads and the second plurality of sequence reads as somatic or germline at step 1502 may be performed as described herein, for example as described with regard to FIG. 2. In an embodiment, at least two variants of the plurality of variants are classified as somatic. It is also understood that the determination of the MAF ratio (step 1503) may be determined for at least one variant of the plurality of variants classified as somatic and based on a first MAF and a second MAF. The first MAF may be determined using variants in the first plurality of sequence reads at a time prior to a treatment and the second MAF may be determined using the same variants in the second plurality of sequence reads at a time after treatment. A first MAF and a second MAF may be determined for the same variant in both the first plurality of sequence reads and the second plurality of sequence reads. It is further understood that the determination of the weighted mean of the MAF ratios (step 1504) may be for the subject. Additionally, it is understood that the determination of the confidence interval associated with the weighted mean of the MAF ratios (step 1505) may be based on the weighted mean of the MAF ratios. Lastly, it is understood that the weighted mean of the MAF ratios and the confidence interval may be outputted as a molecular response score.

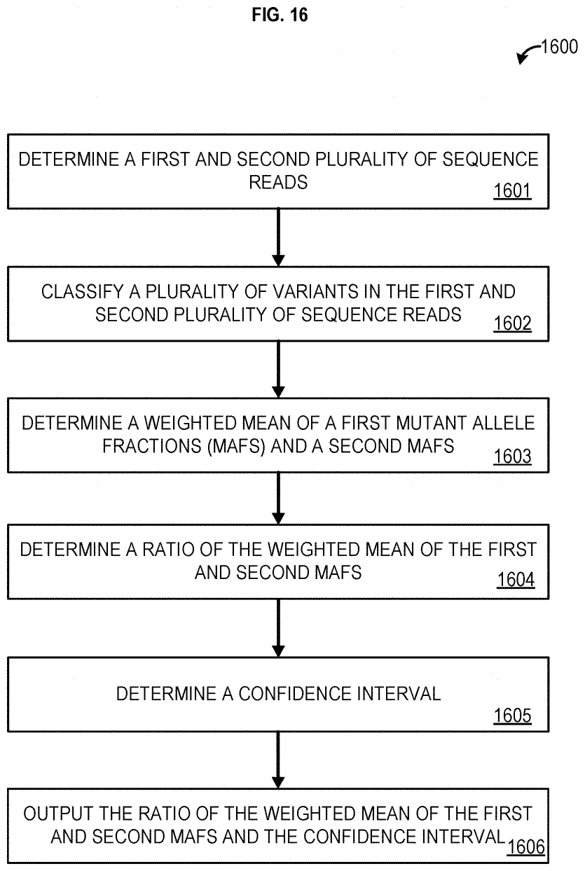

FIG. 16 is a flow chart that schematically depicts a method 1600 that includes determining a first plurality of sequence reads and a second plurality of sequence reads associated with a subject (step 1601). As additionally shown, method 1600 also includes classifying a plurality of variants in the first plurality of sequence reads and the second plurality of sequence reads as somatic or germline (step 1602), determining a weighted mean of the first MAFs and a weighted mean of the second MAFs (step 1603), determining a ratio of the weighted mean of the first MAFs and the weighted mean of the second MAFs (step 1604), determining a confidence interval (step 1605), and outputting, the ratio of the weighted mean of the first MAFs and the weighted mean of the second MAFs and the confidence interval (step 1606). It is understood that the first plurality of sequence reads may be determined before administering the therapy and the second plurality of sequence reads may be determined after administering the therapy. Classifying the plurality of variants in the first plurality of sequence reads and the second plurality of sequence reads as somatic or germline at step 1602 may be performed as described herein, for example as described with regard to FIG. 2. In an embodiment, at least two variants of the plurality of variants are classified as somatic. The first MAF may be determined using variants in the first plurality of sequence reads at a time prior to a treatment and the second MAF may be determined using the same variants in the second plurality of sequence reads at a time after treatment. A first MAF and a second MAF may be determined for the same variant in both the first plurality of sequence reads and the second plurality of sequence reads. It is also understood that the determination of the weighted mean of the first MAFs and the weighted mean of the second MAFs (step 1603) may be determined for at least one variant of the plurality of variants classified as somatic and based on the first MAF and the second MAF. It is further understood that the determination of the ratio of the weighted mean of the first MAFs and the weighted mean of the second MAFs (step 1604) may be for the subject. Additionally, it is understood that the determination of the confidence interval (step 1605) may be based on the ratio of the weighted mean of the first MAFs and the weighted mean of the second MAFs. Lastly, it is understood that the ratio of the weighted mean of the first MAFs and the weighted mean of the second MAFs and the confidence interval may be outputted as a molecular response score.

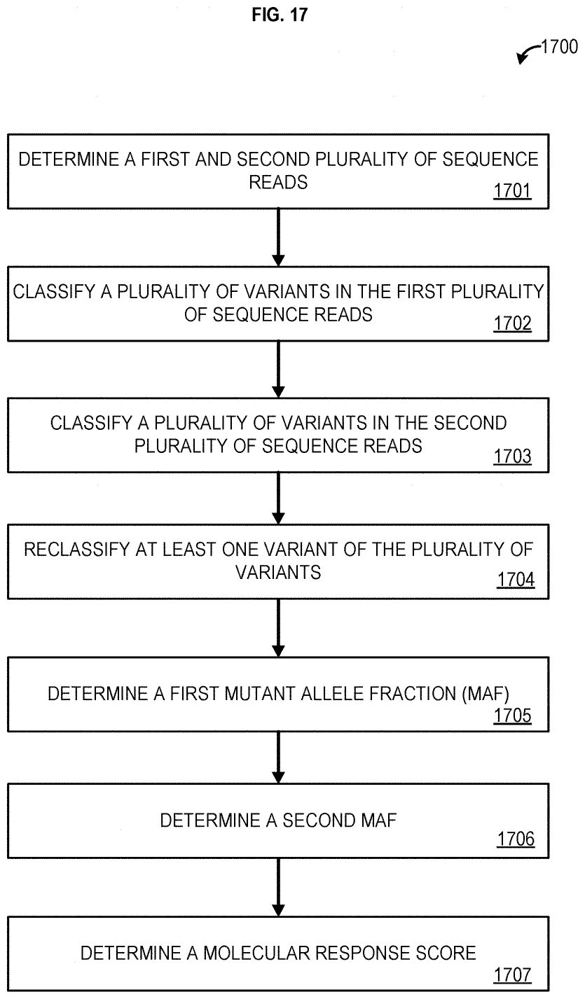

FIG. 17 is a flowchart that schematically depicts a method 1700 that includes determining a first plurality of sequence reads and a second plurality of sequence reads associated with a subject (step 1701). As additionally shown, method 1700 also includes classifying a plurality of variants in the first plurality of sequence reads as somatic or germline (step 1702), classifying the plurality of variants in the second plurality of sequence reads as somatic or germline (step 1703), reclassifying at least one variant of the plurality of variants to resolve a classification discrepancy between the first plurality of sequence reads and the second plurality of sequence reads (step 1704), determining a first mutant allele fraction (MAF) (step 1705), determining a second MAF (step 1706), and determining a molecular response score (1707). It is understood that the first plurality of sequence reads may be determined before administering a therapy and the second plurality of sequence reads may be determined after administering the therapy. Classifying the plurality of variants in the first plurality of sequence reads and the second plurality of sequence reads as somatic or germline at step 1703 may be performed as described herein, for example as described with regard to FIG. 2. The first MAF may be determined using variants in the first plurality of sequence reads at a time prior to a treatment and the second MAF may be determined using the same variants in the second plurality of sequence reads at a time after treatment. A first MAF and a second MAF may be determined for the same variant in both the first plurality of sequence reads and the second plurality of sequence reads. In an embodiment, at least two variants of the plurality of variants are classified as somatic. It is also understood that the determination of the first MAF (step 1705) may be for at least one variant of the plurality of variants classified as somatic and based on at least a portion of the first plurality of sequence reads. It is further understood that the determination of the second MAF (step 1706) may be for at least one variant of the plurality of variants classified or reclassified as somatic and based on at least a portion of the second plurality of sequence reads. Lastly, it is understood that the molecular response may be determined based on the first MAF and the second MAF.

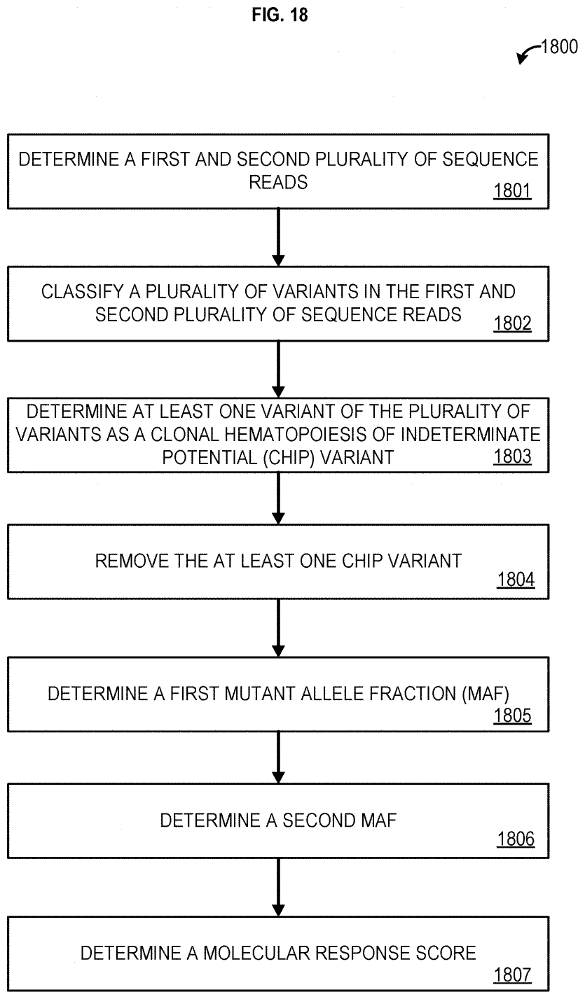

FIG. 18 is a flowchart that schematically depicts a method 1800 that includes determining a first plurality of sequence reads and a second plurality of sequence reads associated with a subject (step 1801). As additionally shown, method 1800 also includes classifying a plurality of variants in the first plurality of sequence reads and the second plurality of sequence reads as somatic or germline (step 1802), determining at least one variant of the plurality of variants as a Clonal Hematopoiesis of Indeterminate Potential (CHIP) variant (step 1803), removing the at least one CHIP variant (step 1804), determining a first mutant allele fraction (MAF) (step 1805), determining a second MAF (step 1806), and determining a molecular response score (step 1807). It is understood that the first plurality of sequence reads may be determined before administering a therapy and the second plurality of sequence reads may be determined after administering the therapy. Classifying the plurality of variants in the first plurality of sequence reads and the second plurality of sequence reads as somatic or germline at step 1802 may be performed as described herein, for example as described with regard to FIG. 2. In an embodiment, at least two variants of the plurality of variants are classified as somatic. The first MAF may be determined using variants in the first plurality of sequence reads at a time prior to a treatment and the second MAF may be determined using the same variants in the second plurality of sequence reads at a time after treatment. A first MAF and a second MAF may be determined for the same variant in both the first plurality of sequence reads and the second plurality of sequence reads. It is also understood that the removal of the at least on CHIP variant (step 1804) may be from the plurality of variants. It is further understood that the determination of the first MAF (step 1805) may be for at least one variant of the plurality of variants classified as somatic and based on at least a portion of the first plurality of sequence reads. Additionally, it is understood that the determination of the second MAF (step 1806) may be for at least one variant of the plurality of variants classified as somatic and based on at least a portion of the second plurality of sequence reads. Lastly, it is understood that the determination of the molecular response score (step 1807) may be based on the first MAF and the second MAF.

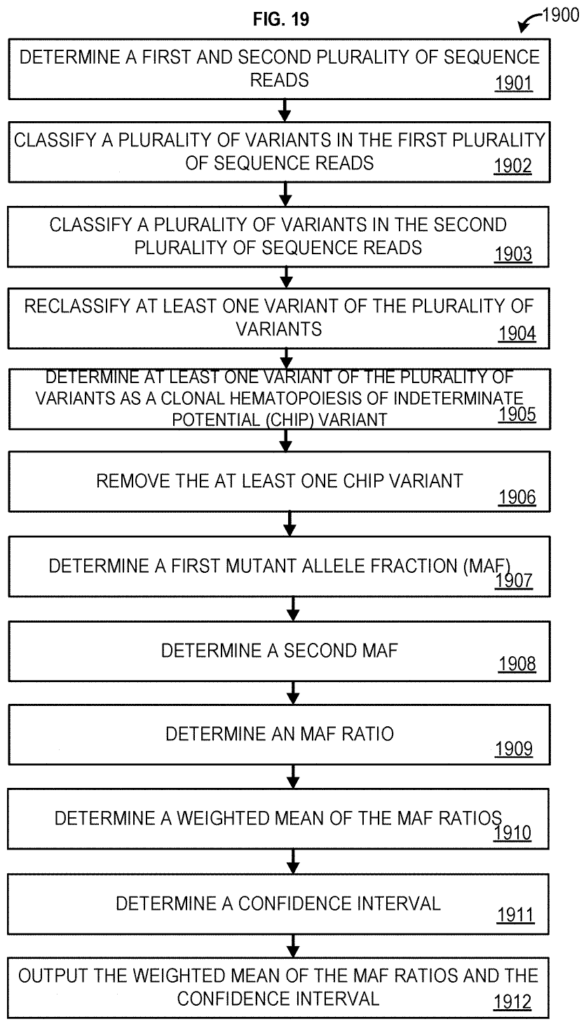

FIG. 19 is a flowchart that schematically depicts a method 1900 that includes determining a first plurality of sequence reads and a second plurality of sequence reads associated with a subject (step 1901). As additionally shown, method 1900 also includes classifying a plurality of variants in the first plurality of sequence reads as somatic or germline (step 1902), classifying the plurality of variants in the second plurality of sequence reads as somatic or germline (step 1903), reclassifying at least one variant of the plurality of variants to resolve a classification discrepancy between the first plurality of sequence reads and the second plurality of sequence reads (step 1904), determining at least one variant of the plurality of variants as a Clonal Hematopoiesis of Indeterminate Potential (CHIP) variant (step 1905), removing the at least one CHIP variant (step 1906), determining a first mutant allele fraction (MAF) (step 1907), determining a second MAF (step 1908), determining an MAF ratio (step 1909), determining a weighted mean of the MAF ratios (step 1910), determining a confidence interval associated with the weighted mean of the MAF ratios (step 1911), and outputting the weighted mean of the MAF ratios and the confidence interval (step 1912). It is understood that the first plurality of sequence reads may be determined before administering a therapy and the second plurality of sequence reads may be determined after administering the therapy. Classifying the plurality of variants in the first plurality of sequence reads at step 1902 and classifying the second plurality of sequence reads as somatic or germline at step 1903 may be performed as described herein, for example as described with regard to FIG. 2. In an embodiment, at least two variants of the plurality of variants are classified as somatic. It is also understood that the removal of the at least one CHIP variant (step 1906) may be from the plurality of variants. The first MAF may be determined using variants in the first plurality of sequence reads at a time prior to a treatment and the second MAF may be determined using the same variants in the second plurality of sequence reads at a time after treatment. A first MAF and a second MAF may be determined for the same variant in both the first plurality of sequence reads and the second plurality of sequence reads. A classification discrepancy may be a variant classified as somatic in the first plurality of sequence reads and as germline in the second plurality of sequence reads. A classification discrepancy may be a variant classified as germline in the first plurality of sequence reads and as somatic in the second plurality of sequence reads. It is further understood that the determination of the first MAF (step 1907) may be for at least one variant of the plurality of variants classified or reclassified as somatic and based on at least a portion of the first plurality of sequence reads. Additionally, it is understood that the determination of the second MAF (1908) may be for at least one variant of the plurality of variants classified or reclassified as somatic and based on at least a portion of the second plurality of sequence reads. It is also understood that the determination of the MAF ratio (1909) may be for at least one variant of the plurality of variants classified or reclassified as somatic and based on the first mutant allele fraction and the second mutant allele fraction. It is further understood that the determination of the MAF ratios (step 1910) may be for the subject. Additionally, it is understood that the determination of the confidence interval associated with the weighted mean of the MAF ratios (step 1911) may be based on the weighted mean of the MAF ratios. Lastly, it is understood that the weighted mean of the MAF ratios and the confidence interval may be outputted as a molecular response score.

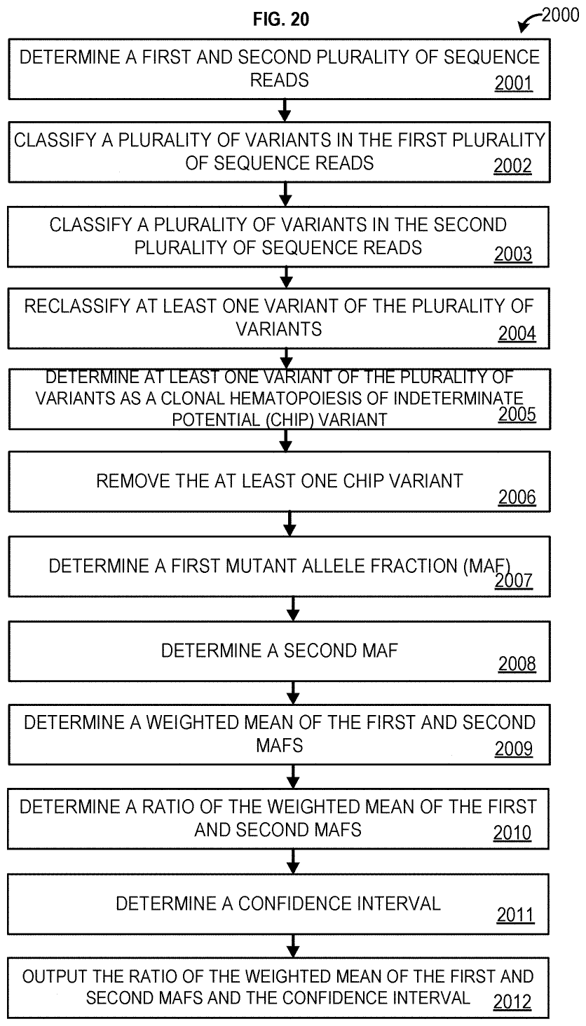

FIG. 20 is a flowchart that schematically depicts a method 2000 that includes determining a first plurality of sequence reads and a second plurality of sequence reads associated with a subject (step 2001). As additionally shown, method 2000 also includes classifying a plurality of variants in the first plurality of sequence reads as somatic or germline (step 2002), classifying the plurality of variants in the second plurality of sequence reads as somatic or germline (step 2003), reclassifying at least one variant of the plurality of variants to resolve a classification discrepancy between the first plurality of sequence reads and the second plurality of sequence reads (step 2004), determining at least one variant of the plurality of variants as a Clonal Hematopoiesis of Indeterminate Potential (CHIP) variant (step 2005), removing the at least one CHIP variant (step 2006), determining a first mutant allele fraction (MAF) (step 2007), determining a second MAF (step 2008), determining a weighted mean of the first MAFs and a weighted mean of the second MAFs (step 2009), determining a ratio of the weighted mean of the first MAFs and the weighted mean of the second MAFs (step 2010), determining a confidence interval (2011), and outputting the ratio of the weighted mean of the first MAFs and the weighted mean of the second MAFs and the confidence interval (step 2012). It is understood that the first plurality of sequence reads are determined before administering a therapy and the second plurality of sequence reads are determined after administering the therapy. Classifying the plurality of variants in the first plurality of sequence reads at step 2002 and classifying the second plurality of sequence reads as somatic or germline at step 2003 may be performed as described herein, for example as described with regard to FIG. 2. In an embodiment, at least two variants of the plurality of variants are classified as somatic. It is also understood that the removal of the at least one CHIP variant (step 2006) may be from the plurality of variants. The first MAF may be determined using variants in the first plurality of sequence reads at a time prior to a treatment and the second MAF may be determined using the same variants in the second plurality of sequence reads at a time after treatment. A first MAF and a second MAF may be determined for the same variant in both the first plurality of sequence reads and the second plurality of sequence reads. It is further understood that the determination of the first MAF (step 2007) may be for at least one variant of the plurality of variants classified or reclassified as somatic and based on at least a portion of the first plurality of sequence reads. Additionally, it understood that the determination of the second MAF (step 2008) may be for at least one variant of the plurality of variants classified or reclassified as somatic and based on at least a portion of the second plurality of sequence reads. It is also understood that the determination of the weighted mean of the first MAFs and a weighted mean of the second MAFs (step 2009) may be for at least one variant of the plurality of variants classified as somatic and based on the first MAF and the second MAF. It is further understood that the determination of the ratio of the weighted mean of the first MAFs and the weighted mean of the second MAFs (step 2010) may be for the subject. Additionally, it is understood that the determination of the confidence interval (step 2011) may be based on the ratio of the weighted mean of the first MAFs and the weighted mean of the second MAFs. Lastly, it is understood that the ratio of the weighted mean of the first MAFs and the weighted mean of the second MAFs and the confidence interval may be outputted as a molecular response score.

### III. Cancer and Other Diseases

In certain embodiments, the methods and aspects disclosed herein are used for longitudinal monitoring of patients with a given disease, disorder or condition. The methods disclosed may be used to track the response of a patient to one or more treatments over time. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic (CLL), chronic myeloid (CIVIL), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

### IV. Customized Therapies and Related Administrations

In some embodiments, the methods disclosed herein relate to identifying and administering therapies to patients having a given disease, disorder or condition. Essentially any cancer therapy (e.g., surgical therapy, radiation therapy, chemotherapy, and/or the like) is included as part of these methods. In certain embodiments, the therapy administered to a subject may comprise at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agents targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GALS, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic options for treating specific genetic-based diseases, disorders, or conditions, other than cancer, are generally well-known to those of ordinary skill in the art and will be apparent given the particular disease, disorder, or condition under consideration.

In certain embodiments, the customized therapies described herein are typically administered parenterally (e.g., intravenously or subcutaneously). Pharmaceutical compositions containing the immunotherapeutic agent are typically administered intravenously. Certain therapeutic agents are administered orally. However, customized therapies (e.g., immunotherapeutic agents, etc.) may also be administered by any method known in the art, including, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

### V. Systems and Computer Readable Media

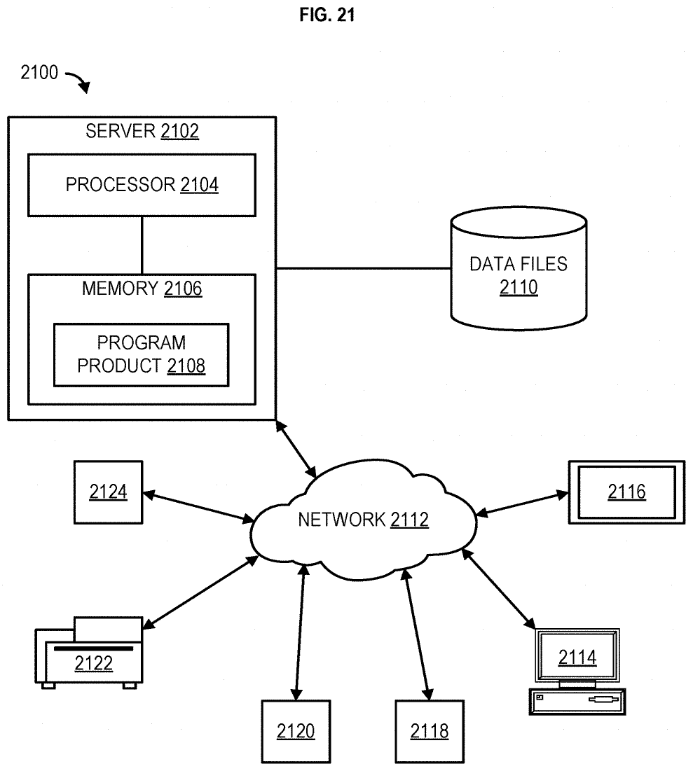

The present disclosure also provides various systems and computer program products or machine readable media. In some embodiments, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 21 provides a schematic diagram of an exemplary system suitable for use with implementing at least aspects of the methods disclosed in this application. As shown, system 2100 includes at least one controller or computer, e.g., server 2102 (e.g., a search engine server), which includes processor 2104 and memory, storage device, or memory component 1506, and one or more other communication devices 2114 and 2116 (e.g., client-side computer terminals, telephones, tablets, laptops, other mobile devices, etc.) positioned remote from and in communication with the remote server 2102, through electronic communication network 2112, such as the internet or other internetwork. Communication devices 2114 and 2116 typically include an electronic display (e.g., an internet enabled computer or the like) in communication with, e.g., server 2102 computer over network 2112 in which the electronic display comprises a user interface (e.g., a graphical user interface (GUI), a web-based user interface, and/or the like) for displaying results upon implementing the methods described herein. In certain embodiments, communication networks also encompass the physical transfer of data from one location to another, for example, using a hard drive, thumb drive, or other data storage mechanism. System 2100 also includes program product 1508 stored on a computer or machine readable medium, such as, for example, one or more of various types of memory, such as memory 2106 of server 2102, that is readable by the server 2102, to facilitate, for example, a guided search application or other executable by one or more other communication devices, such as 2114 (schematically shown as a desktop or personal computer) and 2116 (schematically shown as a tablet computer). In some embodiments, system 2100 optionally also includes at least one database server, such as, for example, server 2110 associated with an online website having data stored thereon (e.g., classifier scores, control sample or comparator result data, indexed customized therapies, etc.) searchable either directly or through search engine server 2102. System 2100 optionally also includes one or more other servers positioned remotely from server 2102, each of which are optionally associated with one or more database servers 2110 located remotely or located local to each of the other servers. The other servers can beneficially provide service to geographically remote users and enhance geographically distributed operations.

As understood by those of ordinary skill in the art, memory 2106 of the server 2102 optionally includes volatile and/or nonvolatile memory including, for example, RAM, ROM, and magnetic or optical disks, among others. It is also understood by those of ordinary skill in the art that although illustrated as a single server, the illustrated configuration of server 2102 is given only by way of example and that other types of servers or computers configured according to various other methodologies or architectures can also be used. Server 2102 shown schematically in FIG. 21, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 2100. As also understood by those of ordinary skill in the art, other user communication devices 2114 and 2116 in these embodiments, for example, can be a laptop, desktop, tablet, personal digital assistant (PDA), cell phone, server, or other types of computers. As known and understood by those of ordinary skill in the art, network 2112 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 2108 is optionally in the form of microcode, programs, cloud computing format, routines, and/or symbolic languages that provide one or more sets of ordered operations that control the functioning of the hardware and direct its operation. Program product 2108, according to an exemplary embodiment, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 2108 implementing the functionality or processes of various embodiments of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 2108 is optionally copied from the computer-readable medium to a hard disk or a similar intermediate storage medium. When program product 2108, or portions thereof, are to be run, it is optionally loaded from their distribution medium, their intermediate storage medium, or the like into the execution memory of one or more computers, configuring the computer(s) to act in accordance with the functionality or method of various embodiments. All such operations are well known to those of ordinary skill in the art of, for example, computer systems.

To further illustrate, in certain embodiments, this application provides systems that include one or more processors, and one or more memory components in communication with the processor. The memory component typically includes one or more instructions that, when executed, cause the processor to provide information that causes sequence information, epigenetic information, classifier scores, cfDNA property data, cfDNA fragment distribution set data, test results, control or comparator results, customized therapies, and/or the like to be displayed (e.g., via communication devices 2114, 2116, or the like) and/or receive information from other system components and/or from a system user (e.g., via communication devices 2114, 2116, or the like).

In some embodiments, program product 2108 includes non-transitory computer-executable instructions which, when executed by electronic processor 2104 perform at least: determining mutant allele frequencies (MAFs) for a plurality of variants from sequence information generated from targeted nucleic acids associated with one or more cancer types in samples obtained from the subject at first and second time points to produce sets of first and second MAFs for at least one variant in the plurality of variants, calculating a ratio of the first and second MAFs for at least one variant in the plurality of variants to produce a set of MAF ratios and a corresponding standard deviation for a MAF ratio in the set of MAF ratios, and calculating a weighted mean of the MAF ratios and a confidence interval to determine the molecular response score for the subject having the cancer. Additional computer readable media embodiments are described herein.

System 2100 also typically includes additional system components that are configured to perform various aspects of the methods described herein. In some of these embodiments, one or more of these additional system components are positioned remote from and in communication with the remote server 2102 through electronic communication network 2112, whereas in other embodiments, one or more of these additional system components are positioned local, and in communication with server 2102 (i.e., in the absence of electronic communication network 2112) or directly with, for example, desktop computer 2114.

In some embodiments, for example, additional system components include sample preparation component 2118 is operably connected (directly or indirectly (e.g., via electronic communication network 2112)) to controller 2102. Sample preparation component 2118 is configured to prepare the nucleic acids in samples (e.g., prepare libraries of nucleic acids) to be amplified and/or sequenced by a nucleic acid amplification component (e.g., a thermal cycler, etc.) and/or a nucleic acid sequencer. In certain of these embodiments, sample preparation component 2118 is configured to isolate nucleic acids from other components in a sample, to attach one or adapters comprising barcodes to nucleic acids as described herein, selectively enrich one or more regions from a genome or transcriptome prior to sequencing, and/or the like.

In certain embodiments, system 2100 also includes nucleic acid amplification component 2120 (e.g., a thermal cycler, etc.) operably connected (directly or indirectly (e.g., via electronic communication network 2112)) to controller 2102. Nucleic acid amplification component 2120 is configured to amplify nucleic acids in samples from subjects. For example, nucleic acid amplification component 2120 is optionally configured to amplify selectively enriched regions from a genome or transcriptome in the samples as described herein.

System 2100 also typically includes at least one nucleic acid sequencer 2122 operably connected (directly or indirectly (e.g., via electronic communication network 2112)) to controller 2102. Nucleic acid sequencer 2122 is configured to provide the sequence information from nucleic acids (e.g., amplified nucleic acids) in samples from subjects. Essentially any type of nucleic acid sequencer can be adapted for use in these systems. For example, nucleic acid sequencer 2122 is optionally configured to perform bisulfite sequencing, pyrosequencing, single-molecule sequencing, nanopore sequencing, semiconductor sequencing, sequencing-by-ligation, sequencing-by-hybridization, or other techniques on the nucleic acids to generate sequencing reads. Optionally, nucleic acid sequencer 2122 is configured to group sequence reads into families of sequence reads, each family comprising sequence reads generated from a nucleic acid in a given sample. In some embodiments, nucleic acid sequencer 2122 uses a clonal single molecule array derived from the sequencing library to generate the sequencing reads. In certain embodiments, nucleic acid sequencer 2122 includes at least one chip having an array of microwells for sequencing a sequencing library to generate sequencing reads.

To facilitate complete or partial system automation, system 2100 typically also includes material transfer component 2124 operably connected (directly or indirectly (e.g., via electronic communication network 2112)) to controller 2102. Material transfer component 2124 is configured to transfer one or more materials (e.g., nucleic acid samples, amplicons, reagents, and/or the like) to and/or from nucleic acid sequencer 2122, sample preparation component 2118, and nucleic acid amplification component 2120.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7th Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

### VI. Examples

### A. Example 1: Comparison of Molecular Response Calculations for Prediction of Patient Outcome

1. Background

Molecular response (MR) estimated as a change in circulating tumor (ctDNA) load between an early on treatment sample (usually 2-9 weeks post treatment start) and pre-treatment baseline has been shown to predict patient response and outcomes across solid tumors and therapy types in many retrospective studies. There is no consensus, however, regarding the best method for assessing molecular response. Therefore, we aimed to assess several molecular response calculations and determine the optimal method for predicting outcomes in individual advanced cancer patients.

2. Method

Aggregate results of >4,000 patient sample pairs (3-10 weeks apart), >1000 patient sample technical replicates, >100 contrived sample dilutions, and in silico simulations were analyzed using cfDNA NGS assay clinical platform (Guardant Health, Inc., Redwood City, Calif., USA). Baseline and on-treatment paired patient samples were collected from advanced cancer patients with over 12 tumor types, including lung, colon, and breast. MR calculations included variant allele fractions (VAFs) of somatic SNVs, indels and fusions. Methods were compared, including Ratio of Maximum VAF (RmaxVAF), Ratio of Mean VAF (RmVAF), and Mean of VAF Ratios (mVAF). Analytical accuracy, reproducibility and limit of detection (LoD) were assessed.

3. Results

Comparison of methods for calculating net change in ctDNA load on >1500 sample pairs showed high correlation (p ranged from 0.93 to 0.98) and categorical agreement split by the median (93%). Therefore selecting an optimal method based on outcome prediction would require prohibitively large patient cohorts. Analytical evaluation and in silico simulations can predict the behavior of each method. Simulations of changes in tumor fraction of real pre-treatment samples found that RmVAF or RmaxVAF are more accurate than mVAFR, which can be skewed by low VAF ratios. Almost 25% of sample pairs have a tumor driver or resistance mutation that is not the maxVAF, suggesting tumor dynamics are better captured by mVAF than maxVAF. Newly-detected on-treatment variants can be an important signal of rising ctDNA levels, impacting MR in approximately 2% of sample pairs.

Importantly, MR accuracy for all methods decreases as maxVAF approaches or falls below the variant LoD, due to both stochastic detection and higher CV of variants at low VAF. Thus the assay variant LoD is a key determinant of the fraction of patients who can receive MR evaluation. Technical replicates identified the variant criteria at which a 50% change in tumor fraction differs significantly from technical variation, and could define analytical reporting limits.

4. Conclusions

Comparison of MR methods in a large set of patient samples and simulations supports RmVAF with inclusion of newly-detected mutations.

### B. Example 2

1. Introduction

Molecular response (MR) is an assessment of the change in circulating tumor (ctDNA) load early on-treatment (usually 3-10 weeks) in comparison to pre-treatment baseline. In many retrospective studies, molecular response was associated with patient response to therapy and long term outcomes across solid tumors and therapy types.

Molecular response has also been shown to predict clinical response earlier than radiographic and/or RECIST response. Multiple methods have been used to calculate molecular response and there is no consensus regarding which method is best.

In this example, several molecular response calculations were assessed and the optimal method for predicting outcomes in individual advanced cancer patients were determined.

2. Methods

Paired samples from >1,500 patient plasma samples spaced 3-10 weeks apart were processed using cfDNA NGS assay clinical platform (Guardant Health, Inc., Redwood City, Calif., USA), with median unique coverage of 4600 molecules sequenced to 20,000× read depth. Somatic and germline SNVs, small indels, and fusions were subset to a 74-cancer associated gene panel space to mimic clinical applications of Molecular Response. >140 patient sample technical replicates were processed on either panel and subsetted to the 74-gene panel space. Three previously published molecular response methods were assessed (see Table 3).

TABLE 3

| Ratio of max VAFs | Ratio of mean VAFs | Mean of VAF ratios |
|---|---|---|
| R(maxVAF) | R(mVAF) | m(rVAF) |
| $\frac{max ( VAF treatment )}{max ( VAF baseline )}$ | $\frac{mean ( VAF treatment )}{mean ( VAF baseline )}$ | $mean [ \frac{{VAF}^{1 … x} treatment}{{VAF}^{1 … x} baseline} ]$ |

3. Results

i. Molecular Response Calculation Captures Changes in ctDNA VAFs of SNVs, Indels, and Fusions

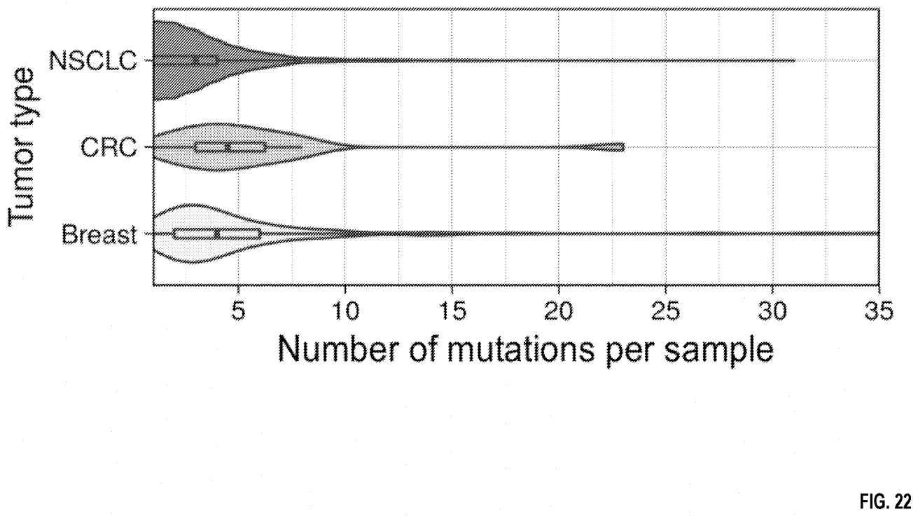

FIG. 22 shows the number of somatic variants detected per sample in a 74-cancer associated gene panel space. The number of somatic SNVs, Indels, and fusions counted towards molecular response calculations per sample for the top 3 cancer types in this study. Median mutation variant count is 4, 5, and 3 for Breast, CRC, and NSCLC, respectively.

ii. Resolution of Somatic Classification with Paired Samples Improves Tumor Signal

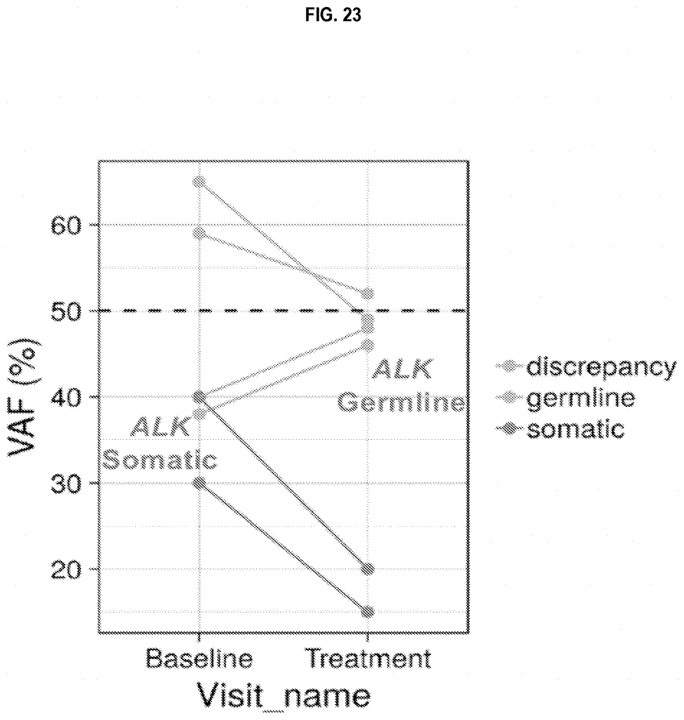

FIG. 23 shows an example of somatic classification discrepancies that could skew MR results. Rare somatic status classification discrepancies (<0.8% of variants) can occur with high tumor fraction and allele imbalance. Unresolved, ALK would skew the MR score against the universally decreasing VAFs.

Table 4 shows an example of resolution of somatic classification discrepancies between patient samples improves variant accuracy. Somatic classification discrepancies in patient sample pairs were resolved by an algorithm based on variant characteristics. Accuracy was assessed against manual resolution by subject matter experts.

TABLE 4

| Accuracy of | Prior to | After resolution, | After resolution, |
|---|---|---|---|
| classification | resolution | VAF-based | MR algorithm |
| Variant-level | 99.2% | 99.3% | 99.8% |
| Patient-level | 87% | 89% | 96% |

iii. Variants are Included in Molecular Response Calculation Based on Detection and VAF Precision

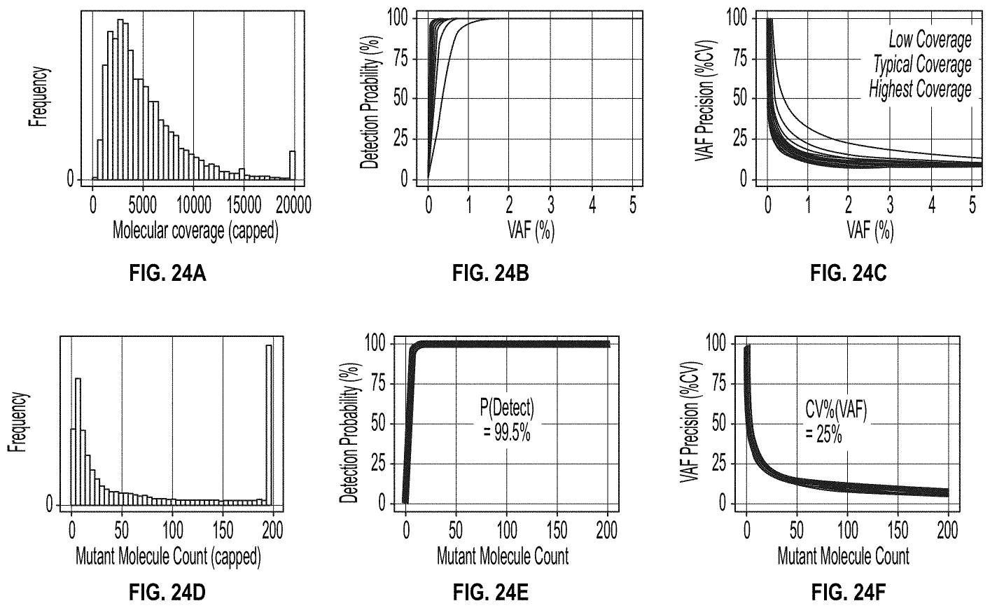

FIG. 24 shows an example of variant precision is determined by Mutant Molecule Count (MMC=VAF*Molecular Coverage). (FIG. 24A) Variants have a range of molecular coverage, depending on sample input and panel design. Probability of variant detection (FIG. 24B) and VAF precision (FIG. 24C) depends on both VAF and molecular coverage (colors, mapping to (FIG. 24A)). MMC (FIG. 24D) is a better metric for variant precision, because it determines the probability of variant detection (FIG. 24E) and VAF precision (FIG. 24F). Variants with low MMC at both timepoints should be excluded from molecular response to better clarify signal from noise.

iv. Molecular Response is Largely Consistent Between Methods but R(mVAF) is More Robust Across Patients

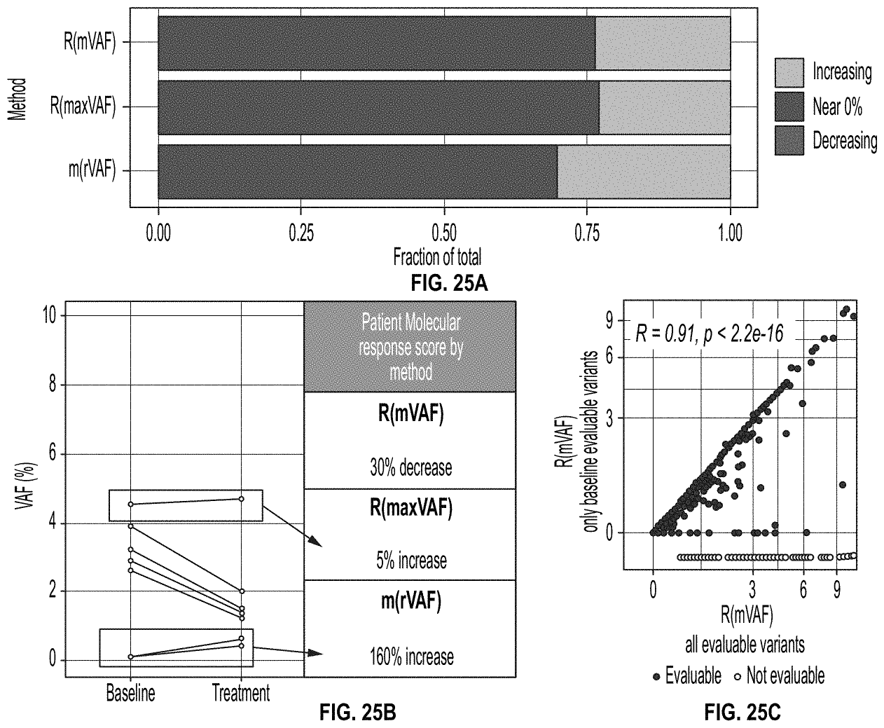

FIG. 25 shows that tumor signal can be outweighed by a minority of variants when using Mean of ratios, m(rVAF), or ratio of max, R(maxVAF). (FIG. 25A) MR score is categorized as Increasing, Decreasing or within precision limit (“Near 0% Change”). Only 8% of patients change between Increasing and Decreasing in any method, showing high categorical correlation (X2 p<0.001). MR correlation ranges from p=0.42 to 0.86 (p<0.001). (FIG. 25B) m(rVAF) is prone to overestimating MR when some VAFs are low (red). R(maxVAF) can be skewed by a single maximum variant (purple) deviating from the majority. 20% of sample pairs have a tumor driver or resistance mutation that is not the maxVAF, suggesting tumor dynamics are better captured by mVAF. (C) Excluding new on-treatment variants would result in a lower MR evaluable rate and excludes signal of emerging variants.

v. Patients with Low Signal of ctDNA Level Change are Identified as not Evaluable for Molecular Response

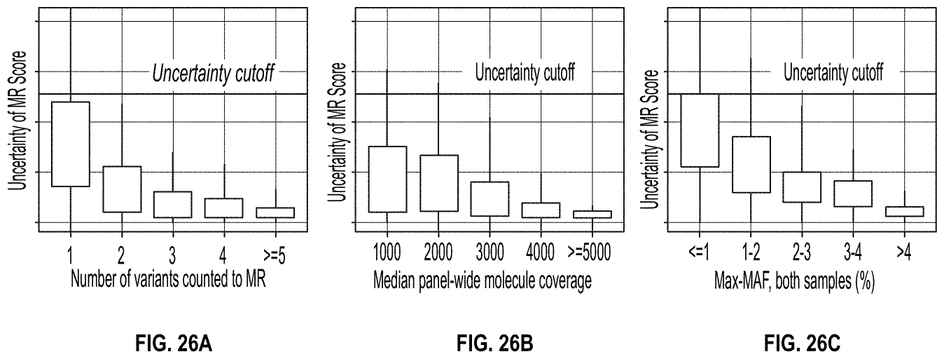

FIG. 26 shows an example that certainty in molecular response score increases with increasing number of variants (FIG. 26A), molecular coverage (FIG. 26B), and maximum VAF (FIG. 26C).

Sample pairs are not evaluable for molecular response using VAF-based methods if there are no somatic variants (approx. 7% of patients), or no somatic variants meeting inclusion criteria (16%). In addition, certainty in molecular response score is calculated theoretically using statistical model of VAF precision. Sample pairs exceeding the acceptable limit of uncertainty (black line) are not evaluable for MR (3%). This results in approx 74% of sample pairs evaluable for MR.

vi. Range of Molecular Response Scores in Clinical Patient Samples Reflect Strong Biological Signal

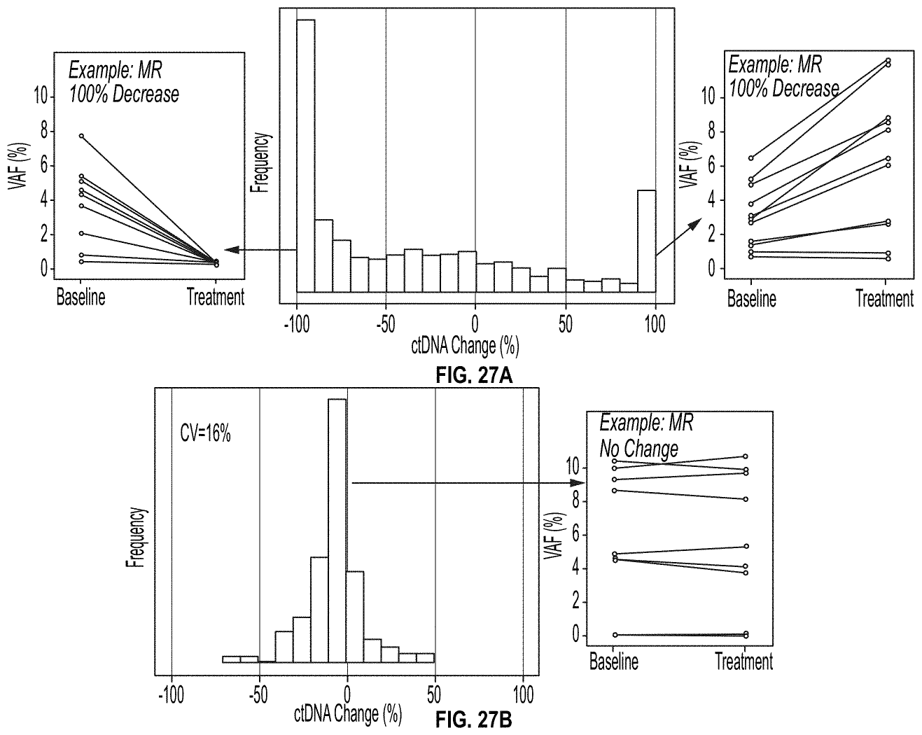

In clinical patient sample pairs, molecular response distribution shows a range of scores from 100% Decrease to >100% Increase (FIG. 27A).

Technical replicates provide null molecular response distribution peaked at 0% change (FIG. 27B).

4. Conclusions

Each component of molecular response calculation is important for accurate assessment of MR, including germline and low-precision variant filtering, overall formulation, and evaluable criteria. Comparison of molecular response methods in a large set of patient samples and simulations supports ratio of mean VAF with inclusion of newly-detected mutations.

### C. Example 3

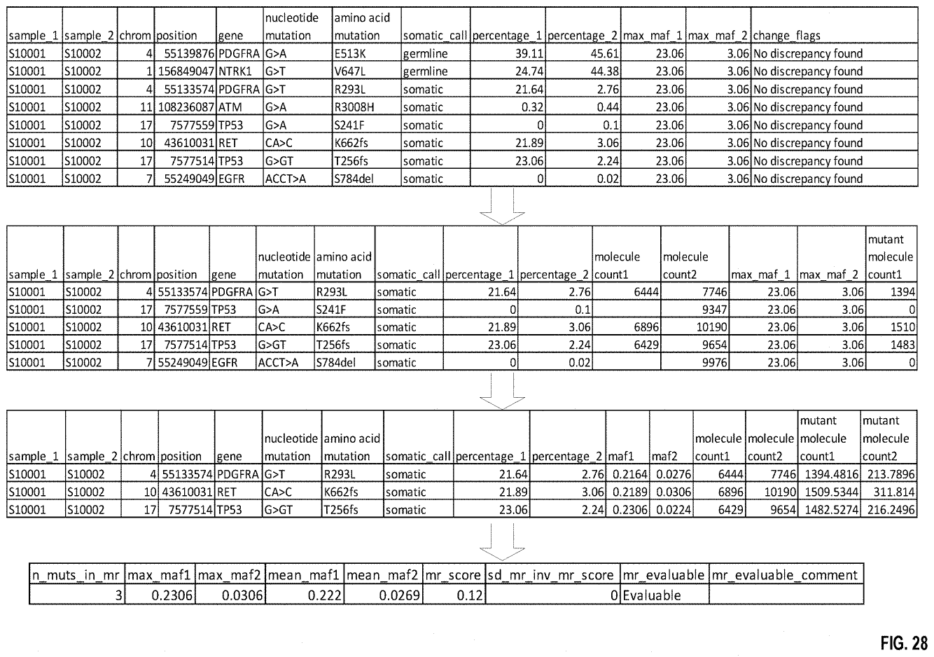

FIG. 28 shows example of a sample pair for MR calculation. Starting with all SNVs, Indels, Fusions detected in either sample, common germline variants are removed. Next, variant somatic/germline classification discrepancies are resolved to give a single classification. (In this example, there were no discrepancies). Next, germline variants are filtered out, and then CHIP variants are filtered out (in this example, ATM.R3008H is a CHIP variant that is removed). Next, variants that do not meet the MMC- or coverage-based inclusion thresholds are removed. In this example, after these filtering steps, three somatic variants (PDGFRA, RET and TP53) remain. Finally, the MR score is calculated from these remaining variants. In this example, the baseline mean VAF is 22.2%, and the on-treatment mean VAF is 2.7%, giving an MR score of 12%, which is a ctDNA Decrease of 88%.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise if different versions of a publication, website or the like are published at different times, the version most recently published at the effective filing date of the application is meant, unless otherwise indicated.

Those skilled in the art will recognize, or be able to ascertain using no more than routine experimentation, many equivalents to the specific embodiments of the method and compositions described herein. Such equivalents are intended to be encompassed by the following claims.

1-46. (canceled)

47. A method for treating a subject having cancer or suspected of having cancer with a therapeutic agent, the method comprising:
(a) determining whether the subject has a molecular response score below a predetermined cutoff point, indicating that the subject is likely a responder to the therapeutic agent, by:
i. obtaining or having obtained a biological sample from the subject, wherein the biological sample comprises cell-free DNA (cfDNA); and
ii. performing or having performed a diagnostic assay on the biological sample to determine the molecular response score of the subject, wherein the diagnostic assay comprises:
a. determining a first plurality of sequence reads and a second plurality of sequence reads associated with a subject having a cancer or suspected of having cancer, wherein the first plurality of sequence reads are determined at a first time point before administering a therapeutic agent and the second plurality of sequence reads are determined at a second time point after administering the therapy;
b. classifying a plurality of variants in the first plurality of sequence reads and the second plurality of sequence reads as somatic or germline;
c. determining, for at least one variant of the plurality of variants classified as somatic, based on a first mutant allele fraction (MAF) at the first time point and a second MAF at the second time point, a first central tendency measure of the first MAFs and a second central tendency measure of the second MAFs;
d. determining a ratio based on the first central tendency measure at the first time point and the second central tendency measure at the second time point;
e. determining a molecular response score from the ratio of the first central tendency measure at the first time point to the second central tendency measure at the second time point;
f. determining the subject is likely responder to the therapeutic agent when the molecular response score is below the predetermined cutoff point likely a responder to the therapeutic agent; and
(b) if the subject is determined to be likely responder, continue administering the therapeutic agent to treat the subject.

48. The method of claim 47, wherein the central tendency measure is one or more of a: mean, median, or mode.

49. The method of claim 47, further comprising determining the subject is a likely non-responder to the therapeutic agent when the molecular response score is at or above the predetermined cutoff point.

50. The method of claim 49, further comprising administering one or more other therapies for the cancer to the subject in view of the molecular response score or discontinuing administering the therapeutic agent to the subject in view of the molecular response score.

51. The method claim 47, further comprising excluding one or more germline and/or clonal hematopoietic variants from the plurality of variants.

52. The method of claim 47, further comprising excluding one or more somatic variants having MAFs that are less than about 0.1%, 0.2%, 0.3%, 0.4%, 0.5%, 0.6%, 0.7%, 0.8%, or 0.9% at both the first and second time points.

53. The method of claim 47, further comprising generating the first plurality of sequence reads and the second plurality of sequence reads from nucleic acid molecules obtained from one or more tissues or cells of the subject.

54. The method of claim 47, further comprising generating the first plurality of sequence reads and the second plurality of sequence reads from cell-free nucleic acids (cfNAs) in samples obtained from the subject, wherein the cfNAs comprise circulating tumor DNA (ctDNA).

55. The method of claim 47, wherein the variants comprise one or more single-nucleotide variants (SNV), insertion/deletion mutations (indels), gene amplifications, and/or gene fusions.

56. The method of claim 47, comprising using a molecule count to calculate the MAF for at least one variant of the plurality of variants.

57. The method of claim 47, further comprising using one or more additional genomic data sources to determine the molecular response score for the subject having the cancer.

58. The method of claim 57, wherein the additional genomic data sources comprise one or more of: a coverage, an off-target coverage, an epigenetic signature, and/or a microsatellite instability score.

59. The method of claim 58, wherein the epigenetic signature comprises a cfNA fragment length, position, and/or endpoint density distribution.

60. The method of claim 58, wherein the epigenetic signature comprises an epigenetic state or status exhibited by one or more epigenetic loci in a given targeted genomic region.

61. The method of claim 60, wherein the epigenetic state or status comprises a presence or absence of methylation, hydroxymethylation, acetylation, ubiquitylation, phosphorylation, sumoylation, ribosylation, citrullination, and/or a histone post-translational modification or other histone variation.

62. The method of claim 47, wherein the therapeutic agent is a chemotherapy drug.

63. The method of claim 47, wherein the therapeutic agent is an immunotherapeutic agent.

64. The method of claim 63, wherein the immunotherapeutic agent is an immune checkpoint inhibitor.

65. The method of claim 47, wherein the second time point is at least 1-24 hours, 1-180 days, 1-12 weeks, 1-25 weeks, or 1-30 weeks after first time point.

66. The method of claim 47, wherein the therapeutic agent is administered at least 30 minutes, 1-2 hours, 1-2 days, 1-2 weeks, or 1-2 months after the first time point.
